(19) United States
(12) Patent Application Publication
(10) Pub. No.: US 2025/0255815 A1
(43) Pub. Date: Aug. 14, 2025

(54) NUCLEIC ACIDS ENCODING A CONSTITUTIVELY-ACTIVE CYCLIC GMP-AMP SYNTHASE AND IMMUNOGENIC DELIVERY VEHICLES FOR SAME

(71) Applicant: Corner Therapeutics, Inc., Watertown, MA (US)

(72) Inventors: Emily GOSSELIN, West Roxbury, MA (US); Dania ZHIVAKI, West Roxbury, MA (US); Jonathan CHOW, Newton, MA (US); Anastasia NIKIFOROV, Watertown, MA (US); Debrup SENGUPTA, Mansfield, MA (US)

(73) Assignee: Corner Therapeutics, Inc., Watertown, MA (US)

(21) Appl. No.: 18/864,457

(22) PCT Filed: May 12, 2023
(86) PCT No.: PCT/US2023/066975

§ 371 Date: Nov. 8, 2024

Related U.S. Application Data
(60) Provisional application No. 63/341,984, filed on May 13, 2022.

(51) Int. Cl. A61K 9/1272; A61K 9/51; A61K 31/4745; A61K 39/00; A61K 39/215; A61K 39/385; A61K 47/26; A61K 47/54; A61P 37/04; C12N 5/00; C12N 5/0784

(52) U.S. Cl. CPC A61K 9/1272; A61K 9/5123; A61K 31/4745; A61K 39/215; A61K 39/385; A61K 47/26; A61K 47/543; A61P 37/04; C12N 5/0018; C12N 5/0639; A61K 2039/53; A61K 2039/55555; A61K 2039/6018

(57) ABSTRACT

The present disclosure relates to compositions for expression of a constitutively-active cyclic GMP-AMP synthase in cells of a mammalian subject and uses thereof for enhancing immunogenicity of mRNA vaccines. The mRNA may be encapsulated in a lipid nanoparticle (LNP) or may be complexed with a lipid (RNA-Lipoplex). The present disclosure also relates to compositions further comprising one or both of a lysophosphatidylcholine (LPC) compound and a pathogen recognition receptor agonist.

### CROSS-REFERENCE TO RELATED APPLICATIONS

This application claims priority to and benefit of U.S. Provisional Patent Application No. 63/341,984, filed May 13, 2022, which is incorporated herein by reference in its entirety.

### SUBMISSION OF SEQUENCE LISTING AS ASCII TEXT FILE

The contents of the electronic sequence listing (165532000240SEQLIST.xml; Size: 22,341 bytes; and Date of Creation: May 11, 2023) is herein incorporated by reference in its entirety.

### FIELD

The present disclosure relates to compositions for expression of a constitutively-active cyclic GMP-AMP synthase in cells of a mammalian subject and uses thereof for enhancing immunogenicity of mRNA vaccines. The mRNA may be encapsulated in a lipid nanoparticle (LNP) or may be complexed with a lipid (RNA-Lipoplex). The present disclosure also relates to compositions further comprising one or both of a lysophosphatidylcholine (LPC) compound and a pathogen recognition receptor agonist.

### BACKGROUND

Advances in mRNA chemistry and delivery systems have enabled the rapid production of several effective mRNA COVID-19 vaccines (Hou et al., Nature Review Materials, 6:1078-1094, 2021). However, a recent report found that T cell reactivity to the SARS-CoV-2 spike protein is considerably lower in vaccinated, uninfected individuals than in individuals with a prior SARS-CoV-2 infection (Naranbhai et al., Cell, 185:1-11, 2022). This is concerning given that higher levels of SARS-CoV-2-reactive CD4+ and CD8+ T cell immune responses were found to be associated with milder COVID-19 disease (Rydyznski Moderbacher et al., Cell, 183(4):996-1012, 2020).

As such, formulations for enhancing cellular immune responses elicited by mRNA vaccines are needed in the art. In particular, adjuvants suitable for increasing adaptive immune responses to nucleic acid-encoded antigens are desirable.

### BRIEF SUMMARY

The present disclosure relates to compositions for expression of a constitutively-active cyclic GMP-AMP synthase in cells of a mammalian subject and uses thereof for enhancing immunogenicity of mRNA vaccines. The mRNA may be encapsulated in a lipid nanoparticle (LNP) or may be complexed with a lipid (RNA-Lipoplex). The present disclosure also relates to compositions further comprising one or both of a lysophosphatidylcholine (LPC) compound and a pathogen recognition receptor agonist.

### BRIEF DESCRIPTION OF THE DRAWINGS

FIG. 1 is an alignment of amino acid sequences of primate cGASΔN proteins including: human (Homo sapiens, SEQ ID NO:1); Rhesus monkey (Macaca mulatta, SEQ ID NO:2); olive baboon (Papio anubis, SEQ ID NO:3); northern white-cheeked gibbon (Nomascus leucogenys, SEQ ID NO:4); common gibbon (Hylobates lar, SEQ ID NO:5); Sumatran orangutan (Pongo abelii, SEQ ID NO:6); chimpanzee (Pan troglodytes, SEQ ID NO:7); western gorilla (Gorilla, SEQ ID NO:8); and a cGASΔN consensus sequence (SEQ ID NO:9).

FIG. 2 is a cartoon depicting activation of the stimulator of interferon genes (STING) innate immune signaling pathway by a cyclic GAMP-AMP synthase (cGAS) that has been engineered to be constitutively-active by removal of its amino-terminal phosphoinositide-binding domain. cGASΔN localizes to mitochondria where it binds DNA resulting in the production of cGAMP.

FIG. 3A-3C depict characteristics of lipid nanoparticles (LNPs) loaded with 22:0 LPC and/or cGASΔN mRNA. FIG. 3A shows that LNPs loaded with 22:0 LPC and/or cGASΔN mRNA are all less than 150 nm in diameter. FIG. 3B shows that mRNA loading efficiency (actual loading compared to theoretical loading) is similar between LNPs loaded with cGASΔN mRNA alone and LNPs loaded with both cGASΔN mRNA and 22:0 LPC. FIG. 3C shows that 22:0 LPC loading efficiency (actual loading compared to theoretical loading) is similar between LNPs loaded with 22:0 LPC alone and LNPs loaded with both cGASΔN mRNA and 22:0 LPC. Data from LNPs loaded with both cGASΔN mRNA and 22:0 LPC ([cGASΔN+22:0 LPC] LNPs) are shown with diagonal stripes.

FIG. 4A-4B show that treatment of murine dendritic cells (DCs) with R848 in combination with LNPs loaded with 22:0 LPC leads to hyperactivation, which is defined by the ability of DCs to secrete IL-1β while remaining viable. Murine bone marrow-derived dendritic cells (BMDCs) were cultured with controls (PBS or R848) or LNPs with or without R848 for 48 hrs. FIG. 4A shows that stimulation of BMDCs with R848 in combination with LNPs loaded with 22:0 LPC ([22:0 LPC] LNPs) or LNPs loaded with both cGASΔN mRNA and 22:0 LPC ([cGASΔN+22:0 LPC] LNPs) induces secretion of IL-1β. In contrast, treatment of BMDCs with LNPs loaded with mRNA encoding cGASΔN alone ([cGASΔN] LNPs) or with [cGASΔN+22:0 LPC] LNPs was not sufficiently stimulatory for induction of IL-1β secretion. FIG. 4B shows the viability of BMDCs as determined via measurement of LDH release when cell culture supernatants were collected. Relative to the PBS control, most treatment conditions resulted in relatively similar levels of cell viability, with the exception of treatment with [cGASΔN+22:0 LPC] LNPs, which caused a reduction in cell viability. Changes in cytokine secretion as a result of various treatment conditions were compared to treatment with R848 alone. p<0.0001****. Statistics were completed using one-way ANOVA with a Tukey post test for multiple comparisons. Data from DCs treated with LNPs loaded with both cGASΔN mRNA and 22:0 LPC ([cGASΔN+22:0 LPC] LNPs) are shown with diagonal stripes.

FIG. 5A-5C shows the effects of various treatments on cytokine secretion by murine DCs. Murine BMDCs were cultured with controls (PBS or R848) or LNPs with or without R848 for 48 hrs. FIG. 5A and FIG. 5B show that stimulation of BDMCs with R848 in the presence or absence of other stimuli induces secretion of IL-6 and TNFα. FIG. 5C and FIG. 5D show that stimulation of BMDCs with R848 in the presence or absence of other stimuli induces secretion of RANTES and IP-10. Interestingly, [cGASΔN] LNPs stimulated secretion of RANTES and IP-10 in the absence of R848, whereas [cGASΔN+22:0 LPC] LNPs in the absence of R848 did not. Changes in cytokine secretion as a result of various treatment conditions were compared to treatment with R848 alone. P<0.05*, p<0.0001****. Statistics were completed using one-way ANOVA with a Tukey post test for multiple comparisons. Data from DCs treated with LNPs loaded with both cGASΔN mRNA and 22:0 LPC ([cGASΔN+22:0 LPC] LNPs) are shown with diagonal stripes.

FIG. 6A-6C shows that human monocyte-derived dendritic cells (moDCs) can simultaneously mediate cGAS/STING signaling and hyperactivation. Human moDCs were cultured with controls (PBS or R848) or LNPs with or without R848 for 48 hrs. FIG. 6A shows that moDCs maintained high levels of viability across all treatment conditions, as determined by measurement of LDH release. FIG. 6B shows that R848 and 22:0 LPC are required for induction of IL-1β secretion by moDCs, and that the addition of cGASΔN mRNA to LNPs was not inhibitory. FIG. 6C shows that treatment of moDCs with LNPs loaded with cGASΔN mRNA stimulates IP-10 secretion even when combined with hyperactivating stimuli R848 and 22:0 LPC. Changes in cytokine secretion as a result of various treatment conditions were compared to treatment with R848 alone. P<0.05*, p<0.0001****. Statistics were completed using one-way ANOVA with a Tukey post test for multiple comparisons. Data from DCs treated with LNPs loaded with both cGASΔN mRNA and 22:0 LPC ([cGASΔN+22:0 LPC] LNPs) are shown with diagonal stripes.

FIG. 7A-7C show that cell surface marker expression by human moDCs is maintained or elevated when hyperactivation is combined with cGAS/STING signaling. Human moDCs were cultured with controls (PBS or R848) or LNPs with or without R848 for 48 hrs before staining for expression of cell surface proteins involved in DC functions. FIG. 7A shows expression of CCR7, a chemokine receptor required for cell migration to lymph nodes, by moDCs as a result of various treatment conditions. FIG. 7B shows expression of CD40, a receptor that engages T cell-expressed CD40L during T cell activation and leads to signaling that further enhances T cell activation, by moDCs as a result of various treatment conditions. FIG. 7C shows expression of CD83, a marker of DC maturation that is involved T cell activation, by moDCs as a result of various treatment conditions. Changes in cell surface marker expression as a result of various treatment conditions were compared to R848 stimulation alone. P<0.05*, p<0.01**, <0.0001****. Statistics were completed using one-way ANOVA with a Tukey post test for multiple comparisons. Data from DCs treated with LNPs loaded with both cGASΔN mRNA and 22:0 LPC ([cGASΔN+22:0 LPC] LNPs) are shown with diagonal stripes.

FIG. 8A-8D show that expression of antigen presenting and co-stimulatory molecules by human moDCs is maintained or elevated when hyperactivation is combined with cGAS/STING signaling. Human moDCs were cultured with controls (PBS or R848) or LNPs with or without R848 for 48 hrs before staining for expression of cell surface proteins involved in DC functions. Treatment of moDCs with various combinations of stimuli resulted in an upregulation of CD80 (FIG. 8A) and CD86 (FIG. 8B) co-stimulatory molecules in comparison to the PBS control. Importantly, moDCs maintained high levels of CD80 and CD86 expression when cGAS/STING signaling was combined with hyperactivation. Treatment of moDCs with various combinations of stimuli resulted in an upregulation of HLA-ABC (FIG. 8C) and HLA-DR (FIG. 8D) in comparison to the PBS control. HLA-ABC and HLA-DR present antigens to CD8+ T cells and CD4+ T cells, respectively. When cGAS/STING signaling was combined with hyperactivation (R848+[cGASΔN+22:0 LPC] LNPs), a further increase in HLA-ABC and HLA-DR expression was observed. Changes in cell surface marker expression as a result of various treatment conditions were compared to R848 stimulation alone. P<0.05*, p<0.0001****. Statistics were completed using one-way ANOVA with a Tukey post test for multiple comparisons. Data from DCs treated with LNPs loaded with both cGASΔN mRNA and 22:0 LPC ([cGASΔN+22:0 LPC] LNPs) are shown with diagonal stripes.

FIG. 9A shows structures of cationic and ionizable lipids suitable for use in the lipid-based mRNA delivery vehicles of the present disclosure. FIG. 9B shows structures of other types of lipids suitable for use in the lipid-based mRNA delivery vehicles of the present disclosure. See also, Hou et al., Nature Review Materials, 6:1078-1094, 2021, which is incorporated herein by reference.

### DETAILED DESCRIPTION

The present disclosure relates to compositions for expression of a constitutively-active cyclic GMP-AMP synthase in cells of a mammalian subject and uses thereof for enhancing immunogenicity of mRNA vaccines. The mRNA may be encapsulated in a lipid nanoparticle (LNP) or may be complexed with a lipid (RNA-Lipoplex). The present disclosure also relates to compositions further comprising one or both of a lysophosphatidylcholine (LPC) compound and a pathogen recognition receptor agonist.

Inducing an inflammatory response can be desirable, such as for an immunotherapy or vaccination. COVID-19 mRNA vaccines (comprising LNPs loaded with mRNA encoding a SARS-CoV-2 antigen) have proven to be effective at reducing frequency and severity of infections. As described herein, inducing the cGAS-STING innate immune pathway adjuvants compositions comprising LNPs loaded with mRNA encoding a protein antigen, which in exemplary embodiments is ovalbumin (OVA). STING signaling is of particular interest out of all possible innate immune signaling pathways because, unlike some other signaling pathways, cGAS-STING activation does not induce translation inhibition.

In order to induce cGAS-STING signaling, a cGAS mutant lacking a portion of its N-terminus was designed such that it is constitutively active (cGASΔN). mRNA encoding the cGAS mutant was packaged in an LNP for cellular uptake and protein expression. The LNP-packaged mRNA encoding cGASΔN has the ability to adjuvant an immune response via the cGAS-STING pathway. Importantly, inclusion of mRNA encoding a constitutively active cGAS, such as cGASΔN, increases the potency of LNPs loaded with mRNA encoding an antigen by increasing inflammatory signals.

### General Techniques and Definitions

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., a molecular weight of about 900 daltons, refers to a molecular weight of from 810 daltons to 990 daltons).

An “effective amount” or a “sufficient amount” of a substance is that amount sufficient to effect beneficial or desired results, including clinical results, and, as such, an “effective amount” depends upon the context in which it is being applied. For instance, in the context of administering an immunogenic composition comprising one or more mRNAs encoding an antigen and a constitutively-active cGAS, an effective amount contains sufficient mRNA, to stimulate an immune response against the antigen (e.g., antigen-reactive antibody and/or cellular immune response).

The terms “individual” and “subject” refer to mammals. “Mammals” include, but are not limited to, humans, non-human primates (e.g., monkeys), farm animals, sport animals, rodents (e.g., mice and rats), and pets (e.g., dogs and cats). In some embodiments, the subject is a human patient, such as a human patient suffering from cancer and/or an infectious disease.

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). As an example, when used in reference to a phospholipid, an isolated phospholipid is at least 90%, 95%, 96%, 97%, 98% or 99% pure as determined by thin layer chromatography, or gas chromatography. As a further example, when used in reference to a recombinant protein, an isolated protein refers to a protein that has been removed from the culture medium of the host cell that produced the protein.

The terms “pharmaceutical formulation” and “pharmaceutical composition” refer to preparations that are in such form as to permit the biological activity of the active ingredient to be effective, and that contain no additional components that are unacceptably toxic to an individual to which the formulation or composition would be administered. Such formulations or compositions are intended to be sterile.

“Excipients” as used herein include pharmaceutically acceptable excipients, carriers, vehicles or stabilizers that are nontoxic to the cell or mammal being exposed thereto at the dosages and concentrations employed. Often the physiologically acceptable excipient is an aqueous pH buffered solution.

The term “antigen” refers to a substance that is recognized and bound specifically by an antibody or by a T cell antigen receptor. Antigens can include peptides, polypeptides, proteins, glycoproteins, polysaccharides, complex carbohydrates, sugars, gangliosides, lipids and phospholipids; portions thereof and combinations thereof. In the context of the present disclosure, the term “antigen” typically refers to a polypeptide encoded by a nucleic acid sequence of a mRNA or a DNA. Polypeptide antigens are preferably at least eight amino acid residues in length and may comprise one or more post-translational modifications.

The term “agonist” is used in the broadest sense and includes any molecule that activates signaling through a receptor. In some embodiments, the agonist binds to the receptor. For instance, a TLR8 agonist binds to a TLR8 and activates a TLR8-signaling pathway.

“Alkyl” refers to monovalent saturated aliphatic hydrocarbyl groups. Cx alkyl refers to an alkyl group having x number of carbon atoms. Cx-Cy alkyl or Cx-y alkyl refers to an alkyl group having between x number and y number of carbon atoms, inclusive.

“Alkylene” refers to divalent saturated aliphatic hydrocarbyl groups.

“Alkenyl” refers to monovalent hydrocarbyl groups having at least one double bond (>C=C<). Cx alkenyl refers to an alkenyl group having x number of carbon atoms. Cx-Cy alkenyl or Cx-y alkenyl refers to an alkenyl group having between x number and y number of carbon atoms, inclusive.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 2-fold to 2,000-fold, or from 5-fold to 500-fold or over, or from 2, 5, 10, 50, or 100-fold to 500, 1,000, 2,000, 5,000, or 10,000-fold.

Conversely, “inhibition” of a response or parameter includes reducing and/or repressing that response or parameter when compared to otherwise same conditions except for a parameter of interest, or alternatively, as compared to another condition (e.g., decrease in abnormal cell proliferation after administration of a composition of the present disclosure, as compared to the administration of a placebo composition or no treatment). For example, “inhibition” of an immune response means a decrease in the response. Depending upon the parameter measured, the decrease may be from 2-fold to 2,000-fold, or from 5-fold to 500-fold or over, or from 2, 5, 10, 50, or 100-fold to 500, 1,000, 2,000, 5,000, or 10,000-fold.

The relative terms “higher” and “lower” refer to a measurable increase or decrease, respectively, in a response or parameter when compared to otherwise same conditions except for a parameter of interest, or alternatively, as compared to another condition. For instance, a “higher level of DC hyperactivation” refers to a level of DC hyperactivation as a consequence of a treatment condition that is at least 2, 3, 4, 5, 6, 7, 8, 9, or 10-fold above a level of DC hyperactivation as a consequence of a control condition. Likewise, a “lower level of DC hyperactivation” refers to a level of DC hyperactivation as a consequence of a treatment condition that is at least 2, 3, 4, 5, 6, 7, 8, 9, or 10-fold below a level of DC hyperactivation as a consequence of a control condition.

As used herein the term “immunization” refers to a process that increases a mammalian subject's immune response to an antigen and therefore improves its ability to resist or overcome infection and/or resist disease.

The term “vaccination” as used herein refers to the introduction of vaccine into a body of a mammalian subject.

“Adjuvant” refers to a substance which, when added to a composition comprising an antigen or a nucleic acid encoding an antigen, enhances or potentiates an immune response to the antigen in the mammalian recipient upon exposure.

The terms “treating”, or “treatment” of a disease refer to executing a protocol, which may include administering one or more therapeutic agents to an individual (human or otherwise), in an effort to obtain beneficial or desired results in the individual, including clinical results. Beneficial or desired clinical results include, but are not limited to, alleviation or amelioration of one or more signs or symptoms of a disease, diminishment of extent of disease, stabilized (i.e., not worsening) state of disease, preventing spread of disease, delay or slowing of disease progression, amelioration or palliation of the disease state, and remission (whether partial or total). “Treatment” also can mean prolonging survival as compared to expected survival of an individual not receiving treatment. Further, “treating” and “treatment” may occur by administration of one dose of a therapeutic agent or therapeutic agents, or may occur upon administration of a series of doses of a therapeutic agent or therapeutic agents. “Treating” or “treatment” does not require complete alleviation of signs or symptoms, and does not require a cure, and specifically includes protocols that have only a palliative effect on the individual. “Palliating” a disease or disorder means that the extent and/or undesirable clinical manifestations of the disease or disorder are lessened and/or time course of progression of the disease or disorder is slowed, as compared to the expected untreated outcome.

The term “constitutively-active” as used herein in reference to cGAS refers to a cGAS variant that binds to DNA in the cytoplasm and catalyzes cGAMP synthesis even under some conditions in which native cGAS has little to no enzymatic activity. In some preferred embodiments, the “constitutively-active cGAS” is a “truncated cGAS”, such as cGASΔN comprising a C-terminal DNA-binding, enzymatic domain in the absence of a N-terminal disordered domain. That is, cGASΔN is a constitutively-active cGAS devoid of regulation of enzymatic activity imparted by the N-terminal disordered domain of full length cGAS.

### I. Constitutively-Active Cyclic GMP-AMP Synthase

Cyclic GMP-AMP synthase, also referred to as cGAMP synthase or cGAS, is an enzymatic sensor of cytosolic DNA. cGAS recognizes double-stranded DNA independent of its sequence resulting in dimerization, formation of liquid-like droplets and production of the secondary messenger 2′3′cyclic GMP-AMP (cGAMP), which binds to and activates STING resulting in expression of interferons and other inflammatory mediators. Human cGAS is 522 amino acids in length, including a N-terminal phosphoinositide-binding domain (residues 1-59) and a C-terminal DNA-binding and enzymatic domain (residues 160-522) (Barnett et al., Cell, 176:1432-1446, 2019). Importantly, expression of cGASΔN in a human leukemia monocytic cell line was found to result in higher levels of expression of interferon and interferon-stimulated genes (Barnett, supra, 2019).

The amino acid sequence of human cGAS (GenBank No. NP_612450.2) is:

(SEQ ID NO: 10)
MQPWHGKAMQRASEAGATAPKASARNARGAPMDPTESPAAPEAALPKAG
KFGPARKSGSRQKKSAPDTQERPPVRATGARAKKAPQRAQDTQPSDATS
APGAEGLEPPAAREPALSRAGSCRQRGARCSTKPRPPPGPWDVPSPGLP
VSAPILVRRDAAPGASKLRAVLEKLKLSRDDISTAAGMVKGVVDHLLLR
LKCDSAFRGVGLLNTGSYYEHVKISAPNEFDVMFKLEVPRIQLEEYSNT
RAYYFVKFKRNPKENPLSQFLEGEILSASKMLSKFRKIIKEEINDIKDT
DVIMKRKRGGSPAVTLLISEKISVDITLALESKSSWPASTQEGLRIQNW
LSAKVRKQLRLKPFYLVPKHAKEGNGFQEETWRLSFSHIEKEILNNHGK
SKTCCENKEEKCCRKDCLKLMKYLLEQLKERFKDKKHLDKFSSYHVKTA
FFHVCTQNPQDSQWDRKDLGLCFDNCVTYFLQCLRTEKLENYFIPEFNL
FSSNLIDKRSKEFLTKQIEYERNNEFPVFDEF.

The amino acid sequence of the N-terminal domain of cGAS is:

(SEQ ID NO: 11)
MQPWHGKAMQRASEAGATAPKASARNARGAPMDPTESPAAPEAALPKAG
KFGPARKSGSRQKKSAPDTQERPPVRATGARAKKAPQRAQDTQPSDATS
APGAEGLEPPAAREPALSRAGSCRQRGARCSTKPRPPPGPWDVPSPGLP
VSAPILVRRDAA.

The amino acid sequence of the C-terminal domain of cGAS (cGASΔN) is:

(SEQ ID NO: 1)
PGASKLRAVLEKLKLSRDDISTAAGMVKGVVDHLLLRLKCDSAFRGVGL
LNTGSYYEHVKISAPNEFDVMFKLEVPRIQLEEYSNTRAYYFVKFKRNP
KENPLSQFLEGEILSASKMLSKFRKIIKEEINDIKDTDVIMKRKRGGSP
AVTLLISEKISVDITLALESKSSWPASTQEGLRIQNWLSAKVRKQLRLK
PFYLVPKHAKEGNGFQEETWRLSFSHIEKEILNNHGKSKTCCENKEEKC
CRKDCLKLMKYLLEQLKERFKDKKHLDKFSSYHVKTAFFHVCTQNPQDS
QWDRKDLGLCFDNCVTYFLQCLRTEKLENYFIPEFNLFSSNLIDKRSKE
FLTKQIEYERNNEFPVFDEF.

The nucleotide sequence encoding human cGASΔN, which was codon-optimized for expression in mouse cells is set forth as SEQ ID NO: 17. A DNA template with this nucleotide sequence was used to prepare mRNA encoding human cGASΔN, which was loaded into LNPs and tested as described in the examples.

Compositions and methods of the present disclosure comprise a nucleic acid encoding a constitutively-active cGAS as a catalytic adjuvant for improving adaptive immune responses elicited by mRNA vaccines. In some preferred embodiments, the constitutively-active cGAS is a truncated cGAS devoid of the N-terminal phosphoinositide-binding domain (cGASΔN). In some preferred embodiments, the constitutively-active cGAS is a truncated cGAS comprising the C-terminal DNA-binding and enzymatic domain (cGASΔN).

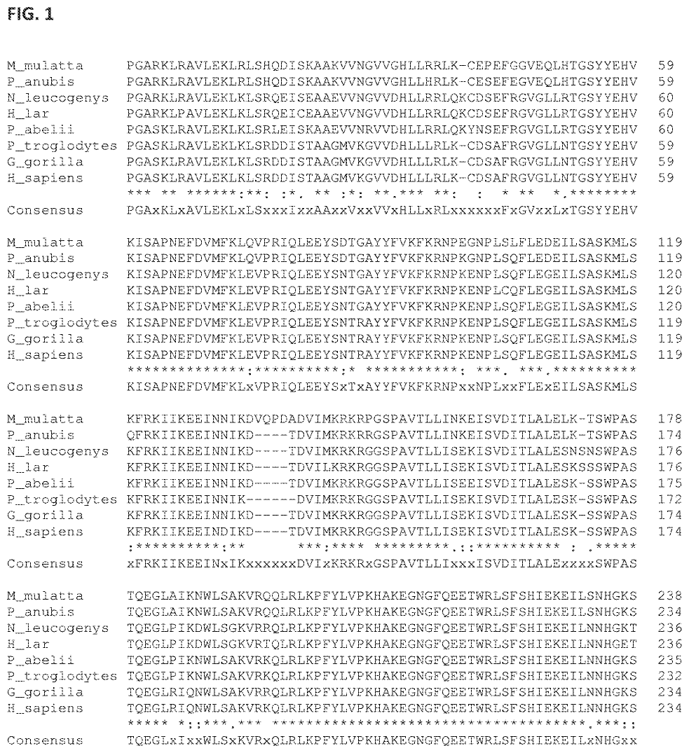

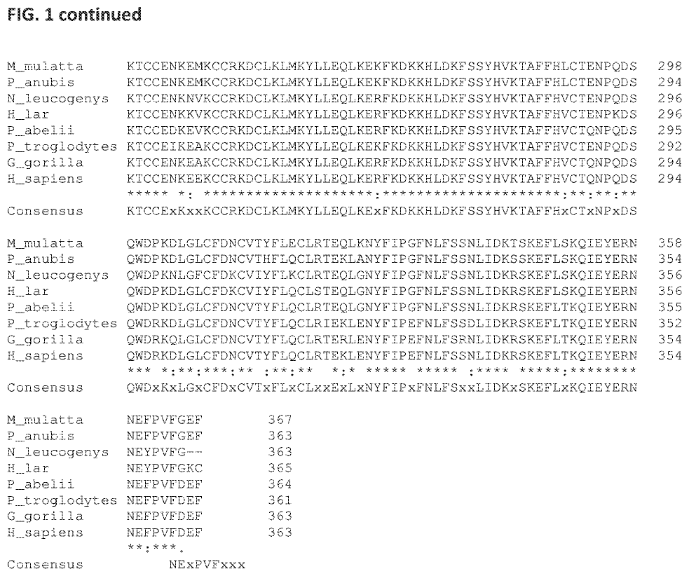

Homologs of cGAS are expressed in species across the animal kingdom, and cGAS amino acid sequences are conserved in higher primates. An alignment of the amino acid sequence of the c-terminal domain of human cGAS with the amino acid sequences of the c-terminal domains of multiple non-human primate cGAS proteins is shown in FIG. 1.

TABLE I
Percent Identity Matrix Created by Clustal 12.1\hat{ }

| # | Species | 1 | 2 | 3 | 4 | 5 | 6 | 7 | 8 |
|---|---|---|---|---|---|---|---|---|---|
| 1 | M._{—}mulatta | 100.00 | 96.42 | 87.53 | 85.95 | 88.71 | 87.26 | 86.78 | 86.50 |
| 2 | P._{—}anubis | 96.42 | 100.00 | 87.53 | 86.23 | 88.98 | 88.09 | 87.88 | 87.33 |
| 3 | N._leucogenys | 87.53 | 87.53 | 100.00 | 96.14 | 93.37 | 89.97 | 90.03 | 90.30 |
| 4 | H._{—}lar | 85.95 | 86.23 | 96.14 | 100.00 | 92.31 | 88.92 | 88.98 | 88.71 |
| 5 | P._{—}abelii | 88.71 | 88.98 | 93.37 | 92.31 | 100.00 | 93.07 | 93.39 | 92.84 |
| 6 | P._troglodytes | 87.26 | 88.09 | 89.97 | 88.92 | 93.07 | 100.00 | 97.51 | 96.95 |
| 7 | H._{—}sapiens | 86.78 | 87.88 | 90.03 | 88.98 | 93.39 | 97.51 | 100.00 | 98.35 |
| 8 | G._{—}gorilla | 86.50 | 87.33 | 90.30 | 88.71 | 92.84 | 96.95 | 98.35 | 100.00 |

\hat{ }Numbers in this table are not SEQ ID NOS.

In some preferred embodiments, the constitutively-active cGAS is a truncated human cGAS devoid of the N-terminal domain (SEQ ID NO:11). In some preferred embodiments, the constitutively-active cGAS is a truncated human comprising the C-terminal domain (SEQ ID NO:1). In some preferred embodiments, cGASΔN comprises the amino acid sequence of SEQ ID NO:1 or the amino acid sequence that is at least 85%, 90%, 95%, 96%, 97%, 98% or 99% identical to SEQ ID NO:1. In some preferred embodiments, cGASΔN comprises the amino acid sequence of SEQ ID NO:2, SEQ ID NO:3, SEQ ID NO:4, SEQ ID NO:5, SEQ ID NO:6, SEQ ID NO:7 or SEQ ID NO:8, or the amino acid sequence at least 95%, 96%, 97%, 98% or 99% identical to SEQ ID NO:2, SEQ ID NO:3, SEQ ID NO:4, SEQ ID NO:5, SEQ ID NO:6, SEQ ID NO:7 or SEQ ID NO:8. In some preferred embodiments, cGASΔN comprises the consensus amino acid sequence of SEQ ID NO:9. For expression in transfected cells, the nucleic acid encoding cGASΔN is in operable combination with a start codon (ATG).

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows: 100 times the fraction X/Y, where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

II. mRNA Encoding an Antigen

Compositions and methods of the present disclosure may comprise an mRNA encoding an antigen or are otherwise suitable for use with a formulation comprising an mRNA encoding an antigen. In some embodiments, the antigen is a proteinaceous antigen. The terms “polypeptide” and “protein” are used interchangeably herein in reference to antigens that comprise peptide chains that are at least 8 amino acids in length. In some embodiments, the antigen is from 8 to 1800 amino acids, 9 to 1000 amino acids, or 10 to 100 amino acids in length. The polypeptide may be post-translationally modified such as by phosphorylation, hydroxylation, sulfonation, palmitoylation, and/or glycosylation.

In some embodiments, the antigen is a tumor antigen that comprises the amino acid sequence of at least one full length protein or fragment thereof. In some embodiments, the tumor antigen comprises an amino acid sequence or fragment thereof from an oncoprotein. In some embodiments, the mammalian antigen is a neoantigen or encoded by a gene comprising a mutation relative to the gene present in normal cells from a mammalian subject. Neoantigens are thought to be particularly useful in enabling T cells to distinguish between cancer cells and non-cancer cells (see, e.g., Schumacher and Schreiber, Science, 348:69-74, 2015). In other embodiments, the tumor antigen comprises a viral antigen, such as an antigen of a cancer-causing virus.

In some embodiments, the tumor antigen is a fusion protein comprising two or more polypeptides, wherein each polypeptide comprises an amino acid sequence from a different tumor antigen or non-contiguous amino acid sequences from the same tumor antigen. In some of these embodiments, the fusion protein comprises a first polypeptide and a second polypeptide, wherein each polypeptide comprises non-contiguous amino acid sequences from the same tumor antigen.

In some embodiments, the antigen is a microbial antigen. In some embodiments, the microbial antigen comprises a viral antigen, a bacterial antigen, a protozoan antigen, a fungal antigen, or combinations thereof. In some embodiments, the microbial antigen comprises a surface protein or other antigenic subunit of a microbe.

In some preferred embodiments, the mRNA comprises a 5′ untranslated region (5′UTR) at the 5′ end of the coding region and a 3′ untranslated region (3′UTR) at the 3′ end of the coding region. In some preferred embodiments, the mRNA comprises one or both of a 5′ cap structure and a polyA tail.

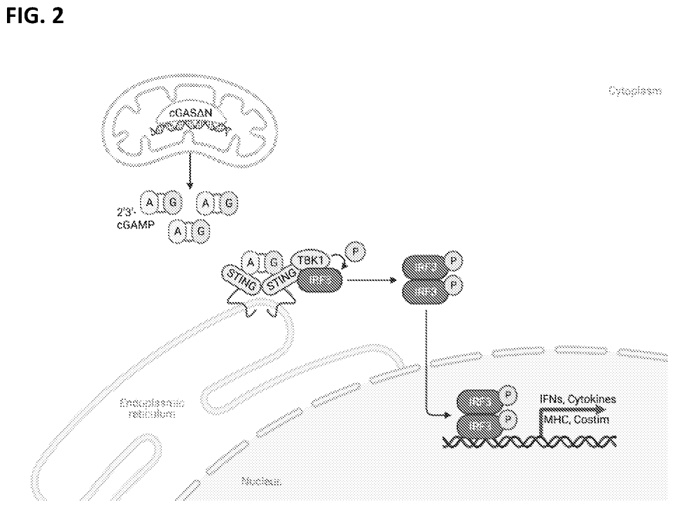

In some embodiments the mRNA further encodes a ribosome skipping sequence, such as the 2A-like (2AL) sequence set forth as SEQ ID NO:12. Additional 2AL sequences are set forth in SEQ ID NO:13, SEQ ID NO:14, and SEQ ID NO:15. In some embodiments in which the mRNA comprises two or more coding regions, the coding regions may be separated by a 2AL sequence. For instance, the 2AL sequence may be located between the coding region of constitutively active cyclic GMP-AMP synthase (cGAS), and the coding region of an antigen (e.g., cGASΔNs-2AL-antigen or antigen-2AL-cGASΔN). Similarly, in some embodiments in which the mRNA comprises coding regions for two or more antigens, the coding regions may be separated by a 2AL sequence (e.g., antigen1-2AL-antigen2). Additional 2AL sequence for use in mRNAs of the present disclosure are known in the art (see, e.g., Luke et al., J. Gen. Virol, 89:1036-1042, 2008, 2AL sequences of FIG. 2 are incorporated herein by reference).

### III. Lipid-Based Delivery Vehicles

Compositions and methods of the present disclosure may comprise a lipid-based delivery vehicle for an mRNA vaccine. In some embodiments, the vehicle is a lipid nanoparticle (LNP). In other embodiments, the vehicle is a lipid that forms a complex with the mRNA (RNA-Lipoplex).

In some embodiments, the LNP comprises at least one lipid selected from the group consisting of an ionizable lipid, a cationic lipid, a phospholipid, a pegylated lipid, a structural lipid, and mixtures thereof. In some embodiments, the at least one lipid comprises an ionizable lipid. In some embodiments, the at least one lipid comprises a cationic lipid. In some embodiments, the at least one lipid comprises a phospholipid. In some embodiments, the at least one lipid comprises a pegylated lipid. In some embodiments, the at least one lipid comprises a structural lipid. In some embodiments, the at least one lipid comprise an ionizable lipid, a phospholipid, a pegylated lipid, and a structural lipid.

In some embodiments, the lipid component of RNA-Lipoplex comprises one or more lipids. In some preferred embodiments, the one or more lipids comprise a first lipid and a second lipid, wherein the first lipid is distinct from the second lipid. In some embodiments, the first lipid is a cationic lipid, and the second lipid is a neutral or anionic lipid.

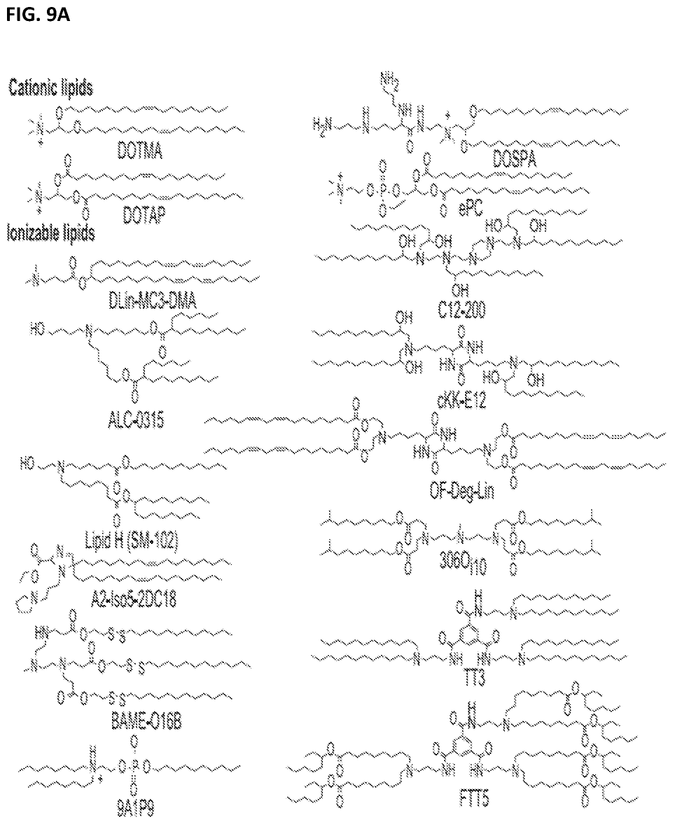

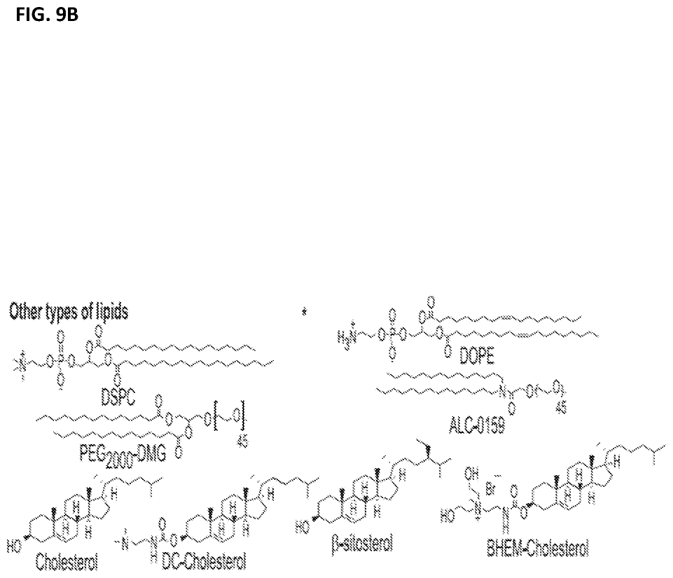

Structures of lipids suitable for use in the lipid-based mRNA delivery vehicles of the present disclosure are depicted in FIG. 9A and FIG. 9B, which are adapted from FIG. 2 of Hou et al., Nature Review Materials, 6:1078-1094, 2021.

### IV. Lysophosphatidylcholine Compounds

Compositions and methods of the present disclosure may comprise a phospholipid, preferably a lysophosphatidylcholine. A “lysophosphatidylcholine” (LPC) or “lysophosphatidylcholine molecule” refers to a glycerol molecule bearing one phosphocholine group on a hydroxyl group of the glycerol and bearing one acyl group on one of the other two hydroxyl groups of the glycerol. The remaining hydroxyl group is unsubstituted.

In some embodiments, the isolated lysophosphatidylcholine (LPC) with a single acyl chain is of the form:

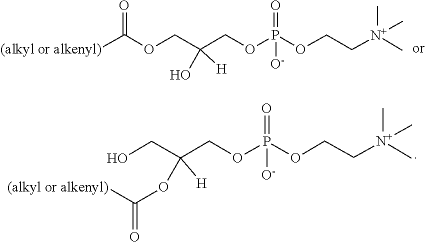

In some embodiments, the isolated lysophosphatidylcholine (LPC) with a single acyl chain is of the form:

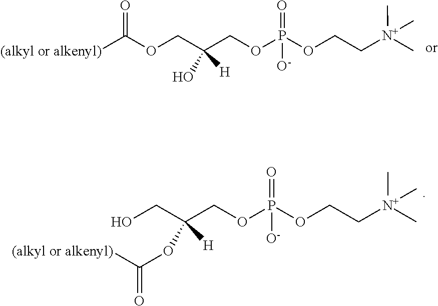

The alkyl or alkenyl chain, together with the carbonyl carbon, forms an acyl chain which is one carbon atom longer than the alkyl or alkenyl chain. For example, a (C23 alkyl)-C(═O)— group forms a C24 acyl chain. Thus, when the group “(alkyl or alkylene)” is a C12-C23 alkyl group (such as a C12-C19 alkyl group or a C20-C23 alkyl group), the (C12-C23 alkyl-C(═O)— group forms a C13-C24 acyl chain (such as a C13-C20 acyl chain or a C21-C24 acyl chain). When the group “(alkyl or alkylene)” is a C12-C23 alkenyl group (such as a C12-C19 alkenyl group or a C20-C23 alkenyl group), the (C12-C23 alkenyl-C(═O)— group forms a C13-C24 acyl chain (such as a C13-C20 acyl chain or a C21-C24 acyl chain). Acyl chains can be referred to as saturated acyl or unsaturated acyl to distinguish between alkyl-containing and alkenyl-containing acyl groups. Standard delta notation or omega notation can be used to indicate the position of one or more double bonds in an unsaturated acyl chain.

Lysophosphatidylcholine (LPC) compounds of the present disclosure have a single acyl chain in which the acyl chain is a C13-C22 acyl chain or a C13-C24 acyl chain. In some embodiments, the acyl chain is a C18-C22 acyl chain or a C21-C24 acyl chain. In some preferred embodiments, the acyl chain is a C22 acyl chain.

### V. Pathogen Recognition Receptor Agonists

Compositions and methods of the present disclosure may comprise a further pathogen recognition receptor (PRR) agonist. In some embodiments, the PRR agonist comprises an agonist of a toll-like receptor (TLR), a NOD-like receptor (NLR), a RIG-I-like receptor (RLR), or a C-type lectin receptor (CLR). In some embodiments, the PRR agonist comprises a TLR7/8 agonist.

### A. TLR7/8 Agonists

The term “TLR7/8 agonist” as used herein refers to an agonist of TLR7 and/or TLR8. In one aspect, the TLR7/8 agonist is a TLR7 agonist. In another aspect, the TLR7/8 agonist is a TLR8 agonist. In a further aspect, the TLR7/8 agonist is an agonist of both TLR7 and TLR8. TLR7/8 agonists of the present disclosure are suitable for hyperactivating human dendritic cells in the presence of LPC.

In some aspects, the TLR7/8 agonist is a small molecule. In some embodiments, the TLR7/8 agonist is a small molecule with a molecule weight of 900 daltons or less, or a salt thereof. That is, the small molecule TLR7/8 agonist is not a large molecule like a recombinant protein or a synthetic oligonucleotide, which is regulatable by the U.S. FDA's Center for Biologics Evaluation and Research. Rather the small molecule TLR7/8 agonist is regulatable by the FDA's Center for Drug Evaluation and Research. In some embodiments, the small molecule has a molecule weight of from about 90 to about 900 daltons. In some embodiments, the TLR7/8 agonist comprises an imidazoquinoline compound. In some preferred embodiments, the TLR7/8 agonist comprises resiquimod (R848).

### B. Other PRR Agonists

In some aspects, the pathogen recognition receptor (PRR) agonist comprises a toll-like receptor (TLR) agonist with the proviso that the TLR agonist does not comprise a TLR7/8 agonist. In some embodiments, the TLR agonist comprises an agonist of one or more of TLR2, TLR3, TLR4, TLR5, TLR9 and TLR13. In some embodiments, the PRR agonist is a TLR2/6 agonist, such as Pam2CSK4. In other embodiments, the TLR agonist is a TLR4 agonist such as monophosphoryl lipid A (MPLA). However, in preferred embodiments, the TLR agonist is not an agonist of TLR2, TLR4 and/or TLR9. For instance, in preferred embodiments, the TLR9 agonist is not a TLR4 ligand such as LPS (endotoxin).

In other aspects, the PRR agonist comprises a NOD-like receptor (NLR) agonist. In further aspects, the PRR agonist comprises a RIG-I-like receptor (RLR) agonist. In additional aspects, the PRR agonist comprises a C-type lectin receptor (CLR) agonist.

### VI. Pharmaceutical Formulations

Some compositions of the present disclosure are pharmaceutical formulations comprising a pharmaceutically acceptable excipient. Pharmaceutical formulations of the present disclosure may be in the form of a solution or a suspension. Alternatively, the pharmaceutical formulations may be a dehydrated solid (e.g., freeze dried or spray dried solid). The pharmaceutical formulations of the present disclosure are preferably sterile, and preferably essentially endotoxin-free. The term “pharmaceutical formulations” is used interchangeably herein with the terms “medicinal product” and “medicament”. In some embodiments, the pharmaceutical formation comprises specific ratios of the various components based on the intended purpose of the formulation.

Pharmaceutically acceptable excipients of the present disclosure include for instance, solvents, buffering agents, tonicity adjusting agents, bulking agents, and preservatives (See, e.g., Pramanick et al., Pharma Times, 45:65-77, 2013). In some embodiments, the pharmaceutical formulations may comprise an excipient that functions as one or more of a solvent, a buffering agent, a tonicity adjusting agent, and a bulking agent (e.g., sodium chloride in saline may serve as both an aqueous vehicle and a tonicity adjusting agent).

In some embodiments, the pharmaceutical formulations comprise an aqueous vehicle as a solvent. Suitable vehicles include for instance sterile water, saline solution, phosphate buffered saline, and Ringer's solution. In some embodiments, the composition is isotonic.

The pharmaceutical formulations may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical formulations may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical formulations may comprise a preservative. Suitable preservatives include for instance antioxidants and antimicrobial agents. However, in preferred embodiments, the pharmaceutical formulation is prepared under sterile conditions and is in a single use container, and thus does not necessitate inclusion of a preservative.

The pharmaceutical formulations of the present disclosure are suitable for parenteral administration. That is the pharmaceutical formulations of the present disclosure are not intended for enteral administration (e.g., not by orally, gastrically, or rectally).

### VII. Methods of Use

In some aspects, the present disclosure relates to methods of use of any one of the compositions or formulations described herein. The methods of use are suitable for a plurality of uses involving stimulating an immune response. In some embodiments, the methods of use comprise methods of treating cancer. In some embodiments, the methods of use comprise methods of inhibiting abnormal cell proliferation. In some embodiments, the methods of use comprise methods of treating or preventing an infectious disease. The methods comprise administering an effective amount of a formulation or a composition described herein to an individual in need thereof to achieve a specific outcome. The individual is a mammalian subject, such as a human patient. In other embodiments, the individual a non-human patient. In some embodiments, the individual is a canine patient. That is in some embodiments, the methods of use involve clinical uses, while in other embodiments the methods of use involve pre-clinical and/or veterinary uses. For preclinical uses, the mammalian subject may be a non-human primate (e.g., monkey or ape) or a rodent (e.g., mouse or rat). For veterinary uses the mammalian subject may be a farm animal (e.g., cow), a sport animal (e.g., horse), a or a pet (e.g., companion animal such as a dog or cat).

### A. Stimulation of an Immune Response

In brief, the present disclosure provides methods of stimulating an immune response in an individual, comprising administering to the individual a composition or formulation described herein in an amount sufficient to stimulate an immune response in the individual. “Stimulating” an immune response (used interchangeably with “eliciting” and immune response), means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response comprises one or more of the group consisting of: stimulating cytokine production; stimulating B lymphocyte proliferation; stimulating interferon pathway-associated gene expression; stimulating chemoattractant-associated gene expression; and stimulating dendritic cell DC maturation. Methods for measuring stimulation of an immune response are known in the art.

For instance, the present disclosure provides methods of inducing an antigen-specific immune response in an individual by administering to the individual a composition or formulation described herein in an amount sufficient to induce an antigen-specific immune response in the individual. In preferred embodiments, the composition or formulation comprises the antigen. In some embodiments, the composition or formulation is administered to a tissue of the individual comprising the antigen. The immune response may comprise one or more of an antigen-specific antibody response, an antigen-specific cytotoxic T lymphocyte (CTL) response, and an antigen-specific helper T (Th) cell response. “Inducing” an antigen-specific antibody response means increasing titer of the antigen-specific antibodies above a threshold level such as a pre-administration baseline titer or a seroprotective level. “Inducing” an antigen-specific CTL response means increasing frequency of antigen-specific CTL found in peripheral blood above a pre-administration baseline frequency. “Inducing” an antigen-specific Th cell response means increasing frequency of antigen-specific Th cells found in peripheral blood above a pre-administration baseline frequency.

Analysis (both qualitative and quantitative) of the immune response can be by any method known in the art, including, but not limited to, measuring antigen-specific antibody production (including measuring specific antibody subclasses), activation of specific populations of lymphocytes such as B cells and helper T cells, production of cytokines such as IFN-alpha, IFN-gamma, IL-6, IL-12 and/or release of histamine. Methods for measuring antigen-specific antibody responses include enzyme-linked immunosorbent assay (ELISA). Activation of specific populations of lymphocytes can be measured by proliferation assays, and with fluorescence-activated cell sorting (FACS). Production of cytokines can also be measured by ELISA. In some embodiments, methods of stimulating an immune response comprise stimulation of interleukin-1beta (IL-1β) secretion, interferon-gamma (IFN-γ) secretion, and/or tumor necrosis factor-alpha (TNF-α) secretion by monocyte-derived dendritic cells or peripheral blood mononuclear cells. In some preferred embodiments, at least 50%, 55%, 60%, 65%, 70% or 75% of the cells contacted with a composition of the present disclosure remain viable at 40-56 hours (or about 48 hours) post-contact.

In some embodiments, the methods are suitable for stimulating an anti-tumor immune response. In other embodiments, the methods are suitable for stimulating an anti-microbe immune response. In some embodiments, the anti-microbe response is an anti-bacterial immune response. In some embodiments, the anti-microbe response is an anti-fungal immune response. In some embodiments, the anti-microbe response is, an anti-viral immune response. In some embodiments, the anti-microbe response is an anti-protozoan immune response.

### B. Treating or Preventing Disease

The present disclosure further provides methods of treating or preventing a disease in an individual, comprising administering to the individual a composition or formulation described herein in an amount sufficient to treat or prevent a disease in the individual. In some embodiments, the disease is cancer. In some embodiments, the disease is abnormal cell proliferation. In other embodiments, the disease is an infectious disease.

In some embodiments, the methods involve treating cancer in an individual or otherwise treating a mammalian subject with cancer. In some embodiments, the cancer is a hematologic cancer, such as a lymphoma, a leukemia, or a myeloma. In other embodiments, the cancer is a non-hematologic cancer, such as a sarcoma, a carcinoma, or a melanoma. In some embodiments, the cancer is malignant.

In some embodiments, the methods involve inhibiting abnormal cell proliferation in an individual. “Abnormal cell proliferation” refers to proliferation of a benign tumor or a malignant tumor. The malignant tumor may be a metastatic tumor.

In some embodiments, the methods involve treating or preventing an infectious disease in an individual. In some embodiments, the infectious disease is caused by a viral infection. In other embodiments, the infectious disease is caused by a bacterial infection. In further embodiments, the infectious disease is caused by a fungal infection. In still further embodiments, the infectious disease is caused by a protozoal infection. Of particular importance are infectious diseases caused by zoonotic pathogens that infect humans as well as other animals such as mammals or birds. In some embodiments, the zoonotic pathogen is transmitted to humans via an intermediate species (vector).

### Enumerated Embodiments

1. A composition comprising an mRNA encapsulated in a lipid nanoparticle (LNP), wherein the mRNA comprises a coding region of a constitutively active cyclic GMP-AMP synthase (cGAS), and the LNP comprises a first phospholipid, and at least one lipid selected from the group consisting of an ionizable lipid, a pegylated lipid, a structural lipid, a second phospholipid, and mixtures thereof, wherein the first phospholipid comprises a lysophosphatidylcholine (LPC) with a single C13-C24 acyl chain.

2. A composition comprising a first mRNA and a second mRNA encapsulated in a lipid nanoparticle (LNP), wherein the first mRNA comprises a coding region of a constitutively active cyclic GMP-AMP synthase (cGAS), the second mRNA comprises a coding region of an antigen; and the LNP comprises a first phospholipid, and at least one lipid selected from the group consisting of an ionizable lipid, a pegylated lipid, a structural lipid, a second phospholipid, and mixtures thereof, wherein the first phospholipid comprises a lysophosphatidylcholine (LPC) with a single C13-C24 acyl chain.

3. A composition comprising an mRNA encapsulated in a lipid nanoparticle (LNP), wherein the mRNA comprises a first coding region and a second coding region separated by a 2A-like sequence, wherein the first coding region is a coding region of a constitutively active cyclic GMP-AMP synthase (cGAS) and the second coding region is a coding region of an antigen or the first coding region is a coding region of an antigen and the second coding region is a coding region of a constitutively active cyclic GMP-AMP synthase (cGAS), and the LNP comprises a first phospholipid, and at least one lipid selected from the group consisting of an ionizable lipid, a pegylated lipid, a structural lipid, a second phospholipid, and mixtures thereof, wherein the first phospholipid comprises a lysophosphatidylcholine (LPC) with a single C13-C24 acyl chain.

4. The composition of any one of embodiments 1-3, wherein the at least one lipid comprises an ionizable lipid, a second phospholipid, a pegylated lipid, and a structural lipid.

5. The composition of any one of embodiments 1-4, wherein the ionizable lipid comprises:
i) 8-[(2-hydroxyethyl)[6-oxo-6-(undecyloxy)hexyl]amino]-octanoic acid, 1-octylnonyl ester (SM-102) or analogs or derivatives thereof, and/or
ii) 6-((2-hexyldecanoyl)oxy)-N-(6-((2-hexyldecanoyl)oxy)hexyl)-N-(4-hydroxybutyl)hexan-1-aminium (ALC-0315) or analogs or derivatives thereof, and/or
iii) (6Z,9Z,28Z,31Z)-heptatriaconta-6,9,28,31-tetraen-19-yl 4-(dimethylamino)butanoate (DLin-MC3-DMA) or analogs or derivatives thereof.

6. The composition of any one of embodiments 1-5, wherein the pegylated lipid is selected from the group consisting of a PEG-modified phosphatidyiethanolamine, a PEG-modified phosphatide acid, a PEG-modified ceramide, a PEG-modified dialkylamine, a PEG-modified diacylglycerol, a PEG-modified dialkylglyerol, and combinations thereof.

7. The composition of any one of embodiments 1-5, wherein the pegylated lipid comprises polyethylene glycol [PEG]2000 dimyristoyl glycerol [DMG].

8. The composition of any one of embodiments 1-7, wherein the structural lipid is selected from the group consisting of cholesterol, fecosterol, sitosterol, ergosterol, campesterol, stigmasterol, brassicasterol, tomatidine, ursolic acid, alpha-tocopherol, and combinations thereof.

9. The composition of any one of embodiments 1-7, wherein the structural lipid comprises cholesterol.

10. The composition of any one of embodiments 1-9, wherein the second phospholipid comprises:
i) a hydrophilic head moiety selected from the group consisting of phosphatidyl choline, phosphatidyl ethanolamine, phosphatidyl glycerol, phosphatidyl serine, phosphatidic acid, 2-lysophosphatidyl choline, and sphingomyelin; and
ii) one or more fatty acid tail moieties selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid.

11. The composition of any one of embodiments 1-9, wherein the second phospholipid is selected from the group consisting of:
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine,
1-oleoyl-2-cholesterylhemisuccinoyl-sn-glycero-3-phosphocholine,
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanolamine (DOPE),
1,2-diphytanoyl-sn-glycero-3-phosphoethanolamine,
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG),
sphingomyelin, and
combinations thereof.

12. The composition of embodiment 11, wherein the second phospholipid comprises 1,2-distearoyl-sn-glycero-3-phosphocholine (DSPC).

13. The composition of any one of embodiments 1-12, wherein the composition further comprises at least one excipient.

14. The composition of embodiment 13, wherein the excipient comprises sucrose.

15. A composition comprising:
i) an mRNA complexed with one or more lipids (RNA-Lipoplex); and
ii) a lysophosphatidylcholine (LPC) with a single C13-C24 acyl chain,
wherein the mRNA comprises a coding region of a constitutively active cyclic GMP-AMP synthase (cGAS), and the one or more lipids comprise a first lipid and a second lipid.

16. A composition comprising:
i) a first mRNA and a second mRNA complexed with one or more lipids (RNA-Lipoplex); and
ii) a lysophosphatidylcholine (LPC) with a single C13-C24 acyl chain;
wherein the first mRNA comprises a coding region of a constitutively active cyclic GMP-AMP synthase (cGAS), and the second mRNA comprises a coding region of an antigen, and the one or more lipids comprise a first lipid and a second lipid.

17. A composition comprising:
i) an mRNA complexed with one or more lipids (RNA-Lipoplex); and
ii) a lysophosphatidylcholine (LPC) with a single C13-C24 acyl chain,
wherein the mRNA comprises a first coding region and a second coding region separated by a 2A-like sequence, the first coding region is a coding region of a constitutively active cyclic GMP-AMP synthase (cGAS) and the second coding region is a coding region of an antigen or the first coding region is a coding region of an antigen and the second coding region is a coding region of a constitutively active cyclic GMP-AMP synthase (cGAS), and the one or more lipids comprise a first lipid and a second lipid.

18. The composition of any one of embodiments 15-17, wherein the first lipid is a cationic lipid, and the second lipid is a neutral or anionic lipid.

19. The composition of embodiment 18, wherein the cationic lipid comprises one or both of:
i) 1,2-di-O-octadecenyl-3-trimethylammonium propane (DOTMA) or analogs or derivatives thereof, and
ii) 1,2-dioleoyl-3-trimethylammonium propane (DOTAP) or analogs or derivatives thereof.

20. The composition of embodiment 18 or embodiment 19, wherein the neutral or anionic lipid comprises:
i) 1,2-di-(9Z-octadecenoyl)-sn-glycero-3-phosphoethanolamine (DOPE) or analogs or derivatives thereof, and/or
ii) cholesterol or analogs or derivatives thereof; and/or
iii) 1,2-dioleoyl-sn-glycero-3-phosphocholine (DOPC) or analogs or derivatives thereof.

21. The composition of any one of embodiments 1-20, wherein the acyl chain of the LPC is a C21-C24 acyl chain.

22. The composition of any one of embodiments 1-20, wherein the acyl chain of the LPC is a C22 acyl chain.

23. The composition of any one of embodiments 1-22, wherein the acyl chain of the LPC is fully saturated.

24. The composition of embodiment 23, wherein the LPC comprises 1-behenoyl-2-hydroxy-sn-glycero-3-phosphocholine [LPC(22:0)].

25. The composition of any one of embodiments 1-24, further comprising a TLR7/8 agonist.

26. The composition of embodiment 25, wherein the TLR7/8 agonist is a small molecule with a molecule weight of 900 daltons or less.

27. The composition of embodiment 26, wherein the TLR7/8 agonist comprises an imidazoquinoline compound.

28. The composition of embodiment 27, wherein the TLR7/8 agonist comprises resiquimod (R848).

29. The composition of any one of embodiments 25-28, wherein the LPC comprises LPC(22:0), and the TLR7/8 agonist comprises resiquimod (R848).

30. The composition of any one of embodiments 1-29, wherein the mRNA or the first mRNA and the second mRNA comprises a 5′ untranslated region (5′UTR) and a 3′ untranslated region (3′UTR).

31. The composition of any one of embodiments 1-30, wherein the mRNA comprises a 5′ cap structure.

32. The composition of any one of embodiments 1-31, wherein the mRNA comprises a polyA tail.

33. The composition of any one of embodiments 1-32, wherein the mRNA is a nucleoside-modified mRNA.

34. A nucleic acid comprising: i) a coding region an antigen, and ii) a coding region of a constitutively-active cyclic GMP-AMP synthase (cGAS), optionally wherein the nucleic acid is mRNA, optionally wherein the nucleic acid is DNA.

35. An expression vector comprising the nucleic acid of embodiment 34.

36. The composition of any one of embodiments 1-35, wherein the constitutively-active cGAS has a greater propensity to self DNA reactivity than its wild-type counterpart.

37. The composition of any one of embodiments 1-36, wherein the cGAS is a truncated cGAS devoid of an amino-terminal phosphoinositide-binding domain (cGASΔN).

38. The composition of embodiment 37, wherein the cGASΔN comprises the amino acid sequence of SEQ ID NO:1 or the amino acid sequence at least 85%, 90%, 91%, 92%, 93%, 94%, 95%, 96%, 97%, 98%, or 99% identical to SEQ ID NO:1.

39. The composition of embodiment 38, wherein the cGASΔN comprises:
(i) the amino acid sequence of SEQ ID NO:2, SEQ ID NO:3, SEQ ID NO:4, SEQ ID NO:5, SEQ ID NO:6, SEQ ID NO:7 or SEQ ID NO:8, or the amino acid sequence at least 95%, 96%, 97%, 98% or 99% identical to SEQ ID NO:2, SEQ ID NO:3, SEQ ID NO:4, SEQ ID NO:5, SEQ ID NO:6, SEQ ID NO:7 or SEQ ID NO:8; or
(ii) the consensus amino acid sequence of SEQ ID NO:9.

40. The composition of embodiment 38, wherein the cGASΔN is encoded by the nucleotide sequence of SEQ ID NO:17.

41. The composition of any one of embodiments 37-40, wherein the coding region of the cGASΔN is in operable combination with a start codon (ATG).

42. The composition of any one of embodiments 1-41, wherein the antigen is a tumor antigen.

43. The composition of embodiment 42, wherein the tumor antigen is a tumor-associated antigen.

44. The composition of embodiment 42, wherein the tumor antigen is a neoantigen.

45. The composition of any one of embodiments 1-41, wherein the antigen comprises a microbial antigen.

46. The composition of embodiment 44, wherein the microbial antigen comprises a viral antigen, a bacterial antigen, a protozoan antigen, or a fungal antigen.

47. The composition of any one of embodiments 1-41, wherein the antigen comprises a surface protein or fragment thereof of a pathogen.

48. The composition of embodiment 47, wherein the pathogen is capable of causing disease in human subjects.

49. The composition of embodiment 47 or embodiment 48, wherein the pathogen is a virus.

50. The composition of embodiment 49, wherein the virus is a SARS-CoV-2.

51. The composition of embodiment 50, wherein the antigen is a spike (S) glycoprotein of the SARS-CoV-2, optionally wherein the spike glycoprotein is a pre-fusion stabilized variant.

52. The composition of any one of embodiments 1-51, wherein the composition does not comprise lipopolysaccharide (LPS) or monophosphoryl lipid A (MPLA).

53. The composition of any one of embodiments 1-52, wherein the composition does not comprise oxidized 1-palmitoyl-2-arachidonoyl-sn-glycero-3-phosphorylcholine (oxPAPC) or a species of oxPAPC.

54. The composition of embodiment 53, wherein the composition does not comprise 2-[[(2R)-2-[(E)-7-carboxy-5-hydroxyhept-6-enoyl]oxy-3-hexadecanoyloxypropoxy]-hydroxyphosphoryl]oxyethyl-trimethylazanium (HOdiA-PC), [(2R)-2-[(E)-7-carboxy-5-oxohept-6-enoyl]oxy-3-hexadecanoyloxypropyl]2-(trimethylazaniumyl)ethyl phosphate (KOdiA-PC), 1-palmitoyl-2-(5-hydroxy-8-oxo-octenoyl)-sn-glycero-3-phosphorylcholine (HOOA-PC), 2-[[(2R)-2-[(E)-5,8-dioxooct-6-enoyl]oxy-3-hexadecanoyloxypropoxy]-hydroxyphosphoryl]oxyethyl-trimethylazanium (KOOA-PC), [(2R)-3-hexadecanoyloxy-2-(5-oxopentanoyloxy)propyl]2-(trimethylazaniumyl)ethyl phosphate (POVPC), [(2R)-2-(4-carboxybutanoyloxy)-3-hexadecanoyloxy propyl]2-(trimethylazaniumyl)ethyl phosphate (PGPC), [(2R)-3-hexadecanoyloxy-2-[4-[3-[(E)-[2-[(Z)-oct-2-enyl]-5-oxocyclopent-3-en-1-ylidene]methyl]oxiran-2-yl]butanoyloxy]propyl]2-(trimethylazaniumyl)ethyl phosphate (PECPC), [(2R)-3-hexadecanoyloxy-2-[4-[3-[(E)-[3-hydroxy-2-[(Z)-oct-2-enyl]-5-oxocyclopentylidene]methyl]oxiran-2-yl]butanoyloxy]propyl]2-(trimethylazaniumyl)ethyl phosphate (PEIPC) and/or 1-palmitoyl-2-azelaoyl-sn-glycero-3-phosphocholine (PAzePC).

55. A pharmaceutical formulation comprising the composition of any one of embodiments 1-54, and a pharmaceutically acceptable excipient.

56. A method for production of hyperactivated dendritic cells, the method comprising contacting the dendritic cells with an effective amount of the composition of any one of embodiments 1-54, any one of embodiments 25-54, or the formulation of embodiment 55 to produce hyperactivated dendritic cells, wherein the hyperactivated dendritic cells secrete IL-1beta without undergoing cell death within about 48 hours of exposure.

57. The method of embodiment 56, wherein the dendritic cells are:
(i) contacted in vivo with the composition; or
(ii) contacted ex vivo with the composition.

58. The method of embodiment 56 or embodiment 57, wherein the hyperactivated dendritic cells:
(i) secrete higher levels of one or more of IL-6, TNFα, RANTES and IP-10 than unstimulated dendritic cells or dendritic cells contacted with empty LNPs; and/or
(ii) express higher levels of at least one cell surface marker selected from the group consisting of CCR7, CD40, CD80, CD83, CD86, MHC class IL, MHC class I, and combinations thereof.

59. A pharmaceutical formulation comprising at least 10\hat{ }3, 10\hat{ }4, 10\hat{ }5 or 10\hat{ }6 of the hyperactivated dendritic cells produced by the method of any one of embodiments 56-58, and a pharmaceutically acceptable excipient.

60. A method of stimulating an immune response against an antigen, comprising administering an effective amount of the pharmaceutical formulation of embodiment 55 or embodiment 59 to an individual in need thereof to stimulate the immune response against the antigen.

61. A method of treating cancer, comprising administering an effective amount of the pharmaceutical formulation of embodiment 55 or embodiment 59 to an individual in need thereof to treat the cancer.

62. A method of inhibiting abnormal cell proliferation, comprising administering an effective amount of the pharmaceutical formulation of embodiment 55 or embodiment 59 to an individual in need thereof to inhibit abnormal cell proliferation.

63. A method of treating or preventing an infectious disease, comprising administering an effective amount of the pharmaceutical formulation of embodiment 55 to an individual in need thereof to treat or prevent the infectious disease.

64. The method of embodiment 63, wherein the infectious disease is a viral disease.

65. The method of embodiment 63, wherein the infectious disease is a bacterial disease.

66. The method or pharmaceutical formulation of any one of embodiments 56-62, wherein the dendritic cells are mammalian cells.

67. The method or pharmaceutical formulation of embodiment 66, wherein the mammalian cells are human cells.

68. The method of any one of embodiments 60-66, wherein the individual is mammal.

69. The method of embodiment 68, wherein the mammal is a human.

70. The method of embodiment 68, wherein the mammal is a dog or a cat.

71. The composition, formulation, or method of any one of embodiments 1-70, wherein the second phospholipid comprises 1,2-distearoyl-sn-glycero-3-phosphocholine (DSPC), and the least one lipid comprises 8-[(2-hydroxyethyl)[6-oxo-6-(undecyloxy)hexyl]amino]-octanoic acid, 1-octylnonyl ester (SM-102) or analogs or derivatives thereof, and cholesterol.

72. The composition, formulation, or method of embodiment 71, wherein the at least one lipid further comprises a pegylated lipid, optionally wherein the pegylated lipid comprises polyethylene glycol [PEG]2000 dimyristoyl glycerol [DMG].

73. The composition, formulation, or method of any one of embodiments 1-72, wherein the LNP has an effective diameter of less than about 250 nanometers, optionally from about 25 to about 250 nanometers, optionally from about 50 to about 200 nanometers, or optionally from about 75 to about 175 nanometers.

74. The composition, formulation, or method of embodiment 73, wherein the LNP has an effective diameter of less than about 200 nanometers.

75. The composition, formulation, or method of embodiment 73, wherein the LNP has an effective diameter of less than about 150 nanometers.

### EXAMPLES

Abbreviations: BMDC (bone marrow-derived dendritic cell); CDS (cytosolic DNA sensor); cyclic GMP-AMP synthase (cGAS); CLR (C-type lectin receptor); DAMP (damage-associated molecular pattern); DC (dendritic cell); dLN (draining lymph node); DLS (dynamic light scattering); DMG-PEG-2000 (polyethylene glycol [PEG]2000 dimyristoyl glycerol [DMG]; DSPC (1,2-distearoyl-sn-glycero-3-phosphocholine); ELSD (evaporative light scattering detector); FLT3L (Fms-related tyrosine kinase 3 ligand); GFP (green fluorescent protein); GV (GenVoy ILM™ formulation); (HOdiA-PC (1-palmitoyl-2-(5-hydroxy-8-oxo-6-octenedioyl)-sn-glycero-3-phosphatidylcholine); HOOA-PC (1-palmitoyl-2-(5-hydroxy-8-oxooct-6-enoyl)-sn-glycero-3-phosphocholine); IFNγ (interferon-gamma); IL-1b/IL1-beta/IL-1β (Interleukin-1beta); KOdiA-PC (1-(palmitoyl)-2-(5-keto-6-octene-dioyl) phosphatidylcholine); KOOA-PC (1-palmitoyl-(5-keto-8-oxo-6-octenoyl)-sn-glycero-3-phosphocholine); LNP (lipid nanoparticle); LPC/Lyso PC (lysophosphatidylcholine); Lyso PC(22:0) (1-behenoyl-2-hydroxy-sn-glycero-3-phosphocholine); LPS (lipopolysaccharide); MC3 ((6Z,9Z,28Z,31Z)-heptatriaconta-6,9,28,31-tetraen-19-yl 4-(dimethylamino)butanoate, also referred to as DLin-MC3-DMA); mcg or μg (microgram); moDC (monocyte-derived dendritic cell); MPLA (monophosphoryl lipid A); NLR (NOD-like receptor); OVA (ovalbumin); oxPAPC (oxidized 1-palmitoyl-2-arachidonyl-sn-glycero-3-phosphorylcholine); PAMP (pathogen-associated molecular pattern); PBMCs (peripheral blood mononuclear cells); PGPC (1-palmitoyl-2-glutaryl-sn-glycero-3-phosphocholine); POVPC (1-palmitoyl-2-(5′-oxo-valeroyl)-sn-glycero-3-phosphocholine); PRR (pathogen recognition receptor); RLR (RIG-I-like receptor); R848 (resiquimod); SC (subcutaneously); STING (stimulator of interferon genes); TNFα (tumor necrosis factor-alpha); and TLR (toll-like receptor).

Although the present disclosure has been described in some detail by way of illustration and example for purposes of clarity and understanding, it will be apparent to those skilled in the art that certain changes and modifications may be practiced. Therefore, the following examples should not be construed as limiting the scope of the present disclosure, which is delineated by the appended claims.

### Example 1: Combination of a Lysophosphatidylcholine (LPC) with a Single Acyl Chain and a TLR7/8 Agonist Hyperactivates Mammalian Peripheral Blood Mononuclear Cells

This example describes the hyperactivation of canine and human peripheral blood mononuclear cells (PBMCs) with a lipid DAMP in combination with a small molecule PAMP.

### Materials and Methods

Isolation of PBMCs from Whole Blood. PBMCs were isolated from whole blood using density gradient centrifugation with Ficoll-Paque PLUS (Cytivia). Whole blood was diluted 1:1 with PBS, layered on top of Ficoll-Paque PLUS and centrifuged at 1000×g for 30 minutes at room temperature. PBMCs were collected, washed twice in PBS, and incubated with Ack lysis buffer (Lonza) to remove any remaining red blood cells.

Cell Culture and Stimulation. Immediately following isolation, PBMCs were plated in RPMI medium containing 10% FBS, 50 units/mL penicillin, 50 mg/mL streptomycin, 2 mM L-glutamine, 1 mM sodium pyruvate, and 50 mM beta-mercaptoethanol (R10 media). Cells were plated at 1×10⁵ (canine cells) or 1×10⁶ (human cells) per well in 96-well flat bottom tissue culture plates. Lyophilized Vaccigrade R848 (Invivogen) was reconstituted and diluted according to manufacturer's recommendations and added to cells at a final concentration of 1 μg/mL. Immediately following, 22:0 LYSO PC was added to cells at a final concentration of 82.5 μM. Additional innate agonists were diluted in R10 media according to manufacturer's recommendations and added to the cells as follows: human GM-CSF (Peprotech) was added at a final concentration of 10 ng/mL; 2′3′ cGAMP (Invivogen) was added at a final concentration of 15 μg/mL; LPS, serotype 055:B5 (Enzo Life Sciences) was added at a final concentration of 1 μg/mL; Alum hydroxide (Invivogen) was added at a final concentration of 30 μg/mL. Cells were incubated at 37° C., 5% CO₂ for two days. Cell cultures were then used for endpoint analyses.

Endpoint Analyses. After culturing PBMCs with PAMPs and DAMPs for two days, supernatant and cell samples were collected for analysis. Cells in culture were pelleted by centrifugation at 400×g for 5 minutes. Half of the media volume in the wells was collected for cytokine quantification by Enzyme-Linked Immunosorbent Assay (ELISA) or Lumit™ Bioluminescent assay, while the remaining media and cells were used to quantify cell viability by assessing metabolic activity.

Quantification of Cytokine Secretion. IL-1β secretion from human PBMCs was assessed using one of the following kits: ELISA MAX Deluxe Set Human IL-1β kit (Biolegend), Invitrogen Human IL-1β kit, or the Lumit™ Human IL-1β Immunoassay (Promega). IFNγ secretion from human PBMCs was assessed using the ELISA MAX Deluxe Set Human IFNγ (Biolegend) and TNFα secretion from human PBMCs was assessed using the Human TNFα Uncoated ELISA kit (Invitrogen). ELISAs were performed according to manufacturer's instructions with the following modifications: i) total sample+buffer volume for incubation was reduced from 100 μL to 50 μL; ii) the top standard was prepared at 500 pg/mL, with two-fold dilutions to 7.8 pg/mL; and iii) sample incubation was completed overnight at 4 C on an orbital shaker. Lumit™ assays were performed according to manufacturer's instructions. IL-1β secretion from canine PBMCs was assessed using the Canine IL-1β/IL-1F2 DuoSet ELISA (R&D) according to manufacturer's instructions with the following modifications: i) total sample+buffer volume for incubation was reduced from 100 μL to 50 μL; ii) sample incubation was completed overnight at 4° C. on an orbital shaker. For all ELISAs, absorbance was measured at 450 nm, with a 570 nm correction, using a Spectramax M5e plate reader (Molecular Devices). For Lumit™ assays, luminescence was measured on all wavelengths using a Spectramax M5e plate reader (Molecular Devices) with an integration time of 500 ms. To determine cytokine concentrations in supernatants, sample concentrations were interpolated using a standard curve via 4PL analysis on GraphPad Prism 9 (GraphPad Software). The interpolated results of samples were then adjusted for any dilutions made to the supernatant.

Quantification of Cell Viability. Cell viability was assessed by quantifying the presence of ATP as an indicator of metabolically active cells using the CellTiter-Glo Luminescent Cell Viability Assay (Promega). Metabolic activity was assessed following manufacturer's instructions. The CellTiter-Glo reagent was mixed with the cell pellets and fresh media then transferred to a white, opaque 96-well plate. Luminescence was measured on all wavelengths on a Spectramax M5e plate reader (Molecular Devices) using an integration time of 500 ms. Percent viability was calculated relative to the control condition of PBMCs treated with R848.

Statistical Analyses. For each condition, cells from each donor were plated for testing in triplicate. For cytokine quantification, triplicate values were used for interpolation and data was plotted as total concentration (pg/mL) or fold change per donor relative to the control condition of R848 alone. For viability quantification, each donor triplicate was averaged, and the average was used as one donor measurement. Multiple donors were tested and each data point on the column graphs represents the value for a donor. To test for differences in test conditions, test results were compared to the control condition of R848 alone. P-values were calculated using a mixed-effects one-way ANOVA, with corrections for multiple comparisons using a Dunnett's test.

Results—Treatment with 22:0 LYSO PC and R848 Hyperactivates Canine PBMCs

The combination of 22:0 LYSO PC (DAMP) and the TLR7/8 agonist R848 (PAMP) was found to have potent hyperstimulatory activity in human moDCs In order to assess whether this hyperstimulatory activity translates to other clinically relevant species, the ability of 22:0 LYSO PC+R848 to hyperactivate PBMCs isolated from canine whole blood was assessed. For each data set, PBMCs from multiple donors were used in lieu of moDCs due to the lack of canine-specific reagents available to induce bone fide canine moDCs. In brief, PBMCs were isolated from whole blood using density gradient centrifugation and then cultured for two days with the hyperactivating stimuli of interest.

After two days in culture, hyperactivation was assessed by quantification of IL-1β in cell culture supernatants and measurement of cell viability. When treated with 22:0 LYSO PC and R848 together, canine PBMCs secreted comparable or higher levels of IL-1β compared to every other stimulus tested, both as concentration per mL, as well as fold change per donor relative to R848 alone. Consistent with previous studies showing that monocytes, which make up 5-10% of PBMCs, can release IL-1β in response to activation with R848, canine PBMCs had elevated levels of IL-1β secretion with R848 alone compared to untreated cells. The pyroptotic combination of LPS+Alum elicited high levels of IL-1β as expected. Notably, while PGPC+R848 elicited similar levels of IL-1β compared to R848 alone, neither GM-CSF nor 2′3′cGAMP induced significant IL-1β secretion from canine PBMCs compared to untreated cells.

Although IL-1β can be detected one day after hyperactivation of canine PBMCs in cell culture supernatants, cell viability was evaluated two days post-hyperactivation to ensure enduring viability after IL-1β secretion. 22:0 LYSO+R848 did not significantly reduce relative cell viability. Interestingly, PGPC in combination with R848 proved to be somewhat toxic to canine PBMCs, although it was not observed to be toxic to human moDCs or human PBMCs. Together these data demonstrate that 22:0 LYSO+R848 elicits high levels of IL-1β secretion from canine PBMCs, which is indicative of hyperactivation.

Results—Treatment with 22:0 LYSO PC and R848 Hyperactivates Human PBMCs

Hyperactivation experiments were also performed with PBMCs isolated from whole blood obtained from human donors. In brief, PBMCs were isolated from whole blood by density gradient centrifugation from multiple human donors and cultured for two days with the hyperactivating stimuli of interest.

Human PBMCs, like human moDCs and canine PBMCs, secreted IL-1β at levels higher or comparable to all other stimuli tested. Similar to canine PBMCs, human PBMCs secreted IL-1β in response to R848 alone due to monocyte activation, and this was elevated by addition of 22:0 LYSO PC. The pyroptotic combination of LPS+Alum elicited high levels of IL-1β as expected. Consistent with observations in canine PBMCs, PGPC+R848 did not induce substantially higher levels of IL-1β than R848 alone. GM-CSF did not induce levels of IL-1β secretion from human PBMCs significantly above background levels produced by untreated cells.

Viability of human PBMCs was also assessed two days post-hyperactivation to ensure enduring viability of human PBMCs after IL-1f secretion. No significant decreases in human PBMC viability were observed after treatment with any of the stimuli. Together these data demonstrate that both human and canine PBMCs are hyperactivated by 22:0 LYSO PC+R848. Interestingly, canine PBMCs are hyperactivated to a greater extent by 22:0 LYSO PC+R848 than by PGPC+R848.

Because activated human PBMCs can secrete other cytokines in addition to IL-1β, the secretion of the pro-inflammatory cytokines IFNγ and TNFα in cell culture supernatants was measured two days post-hyperactivation. The combination of 22:0 LYSO PC+R848 induced the highest fold change per donor in both IFNγ secretion and TNFα secretion relative to R848 alone as compared to all other stimuli tested. Notably, although LPS+Alum induced high levels of IL-1β secretion from human PBMCs, this combination of stimuli did not induce a fold increase in IFNγ or TNFα secretion. Moreover, neither GM-CSF nor 2′3′cGAMP elicited a substantial fold change in IFNγ secretion over R848 alone. These data indicate that the combination of 22:0 LYSO PC+R848 is superior at inducing secretion of the proinflammatory cytokines IFNγ and TNFα from human PBMCs.

### Example 2: Both cGASΔN mRNA and 22:0 LPC can be Loaded into Lipid Nanoparticles (LNPs)

### Materials & Methods

Materials. Lipids for LNPs were purchased from Cayman Chemicals (SM102) or Avanti (22:0 LPC, DSPC, DMG-PEG2000). Cholesterol was purchased from Sigma. cGASΔN mRNA was custom ordered and synthesized via in vitro transcription from a linearized template DNA (Trilink). The human cGASΔN sequence was codon optimized for expression in murine cells, and the synthetic nucleotide sequence is set forth as SEQ ID NO:17. The mRNA sequence was capped using Trilink's proprietary Clean Cap mRNA technology, with a N1-methylpseudouridine base modification, and a 120 residue polyA tail. The sequence contains a Bbs1 restriction enzyme site. The sequence information was used to build plasmids, from which the mRNA was synthetically synthesized. The mRNA was phosphatase treated after synthesis.

LNP Synthesis. Lipid nanoparticles (LNPs) were prepared using a custom LNP lipid mix (Table 2-1). cGASΔN mRNA was prepared at 0.02 mg/mL in sodium citrate buffer, pH 4. The custom LNP lipid mix was prepared at 12.5 mM. Ratio of mRNA/22:0 LPC was chosen based on previous experiments that determined in vitro activity of each component. LNPs were synthesized using the NanoAssemblr Ignite instrument (Precision Nanosystems). Lipids in ethanol were combined with the mRNA solutions individually at a 1:3 volumetric ratio, using a flow rate of 12 mL/min. LNPs were washed in 10 volumes of phosphate buffered saline (PBS), pH 7.4 to remove residual ethanol, and then concentrated using Amicon 10K MWCO centrifugal filters. LNPs were filtered through a 0.2 μm filter before use.

TABLE 2-1
LNP Formulations

| | 22:0 LPC | cGASΔN | 22:0 LPC + |
|---|---|---|---|
| Lipid Mix | LNPs | LNPs | cGASΔN LNPs |
| SM102 | 40% | 40% | 40% |
| DSPC | 0% | 35% | 0% |
| Cholesterol | 28.5% | 28.5% | 28.5% |
| DMG-PEG2000 | 1.5% | 1.5% | 1.5% |
| 22:0 LPC | 35% | 0% | 35% |
| mRNA | N/A | cGASΔN | cGASΔN |

LNP Characterization. Loading of mRNA into LNPs was quantified using a RiboGreen assay (ThermoFisher) following the manufacturer's protocol. Samples were diluted to fall within the range of the standard curve. LNPs were lysed using Triton X-100 to assess encapsulation of mRNA into LNPs. Both total mRNA and encapsulated mRNA were quantified. The size of the LNPs was assessed using dynamic light scattering (DLS) on the NanoBrook Omni (Brookhaven). LNPs were diluted 1:10 in PBS before running on the DLS. Three 90 second measurements were recorded for each sample.

### Results

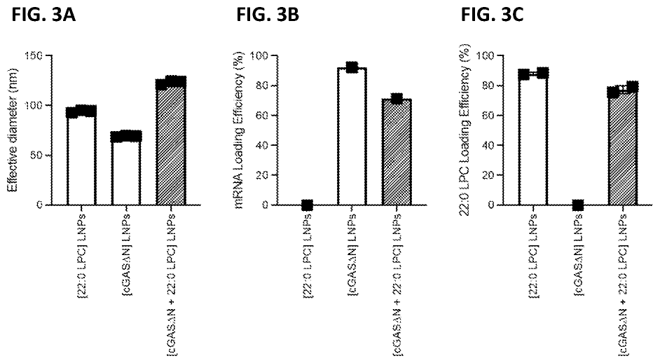

LNPs can be loaded with cGASΔN mRNAs and 22:0 LPC at similar levels to LNPs loaded with either cGASΔN mRNA or 22:0 LPC. All LNPs−22:0 LPC LNPs, cGASΔN LNPs, and cGASΔN+22:0 LPC LNPs—show similar sizes, with all LNPs <150 nm in size (FIG. 3A) with relatively uniform distribution (PDI <0.3). cGASΔN LNPs and cGASΔN+22:0 LPC loaded LNPs showed similar mRNA loading levels, though there was a slight reduction in overall loading efficiency (actual/theoretical loading) when 22:0 LPC was also added to the LNPs (FIG. 3B). 22:0 LPC LNPs and cGASΔN+22:0 LPC LNPs showed similar loading levels and efficiencies (actual/theoretical) for 22:0 LPC (FIG. 3C).

### Example 3: Mouse Dendritic Cells Achieve a Unique Activation State by Treatment with mRNA Encoding cGASΔN and Hyperactivating Stimuli

### Materials & Methods

Generation of murine bone marrow-derived dendritic cells (BMDCs). Leg femur and tibia were removed from mice, cut with scissors, and flushed into sterile tubes. Bone marrow suspension was treated with ACK Lysis Buffer for 1 minute, then passed through a 40 pm cell strainer. Cells were counted and resuspended in media consisting of complete IMDM containing 10% FBS, penicillin and streptomycin, and supplements of L-glutamine and sodium pyruvate (I10). Cells were then plated at 8E6 bone marrow cells per well in a P12 plate. Recombinant mouse FLT3L (Miltenyi) was added to cultures at 200 ng/mL. Differentiated cells were used for subsequent assays on day 9. The efficiency of differentiation was monitored by flow cytometry using a BD Symphony A3, and CD11c⁺MHC-II⁺ cells were routinely above 80% of living cells. For each experiment, 5 to 15 mice were used to generate DCs from bone marrow.

Hyperactivation of murine BMDCs and cGASΔN delivery. BMDC s were harvested on day 9 post differentiation, washed with PBS and re-plated in complete IMDM media (I10) at a concentration of 2×10⁵ cells/well. Cells were cultured in the presence or absence of LNPs loaded with 22:0 LPC LNPs at 100 μM and with or without cGASΔN mRNA at ˜1 μg/mL (Table 3-1). In certain conditions, R848 was added at 1 μg/mL final concentration. Forty-eight hours post stimulation, supernatants were collected for cytokine secretion assessment. IL-1β cytokine secretion by BMDCs was measured using sandwich ELISAs (Invitrogen) following manufacturer's instructions. Additional cytokines were measured using the LEGENDplex Mouse Anti-viral Response Panel (Biolegend) according to manufacturer's instructions. Cell viability was assessed by measuring LDH release into fresh supernatant using the CyQUANT LDH Cytotoxicity Assay (Invitrogen) according to manufacturer's instructions.

TABLE 3-1
In Vitro Dosing of Murine BMDC

| | 22:0 LPC | cGASΔN | 22:0 LPC + | |
|---|---|---|---|---|
| | LNPs | LNPs | cGASΔN LNPs | |
| 22:0 LPC Dose | 100 μM | N/A | 100 | μM |
| mRNA Dose | N/A | 0.91 μg/mL | 0.91 | μg/mL |

### Results

In previous work, we found that DC hyperactivation using R848 and 22:0 LPC activates the transcription factor NF-kB, resulting in the production of inflammatory cytokines such as IL-6 and TNFα. Additionally, the NLRP3 inflammasome is activated, resulting in IL-1β production. Distinct from these cellular responses, cGAS activation and the resulting STING signaling activate the transcription factor IRF3, which co-regulates some genes with NF-kB but also regulates a unique set of genes independent of NF-kB. cGASΔN is a modified cGAS that is constitutively active. Using LNPs formulated with cGASΔN mRNA with and without 22:0 LPC, we hypothesized that cGAS signaling and hyperactivation can induce a complementary set of inflammatory signals.

To test our hypothesis, mouse bone marrow cells were differentiated into DCs using FLT3L. BMDCs were then stimulated with R848, 22:0 LPC, and cGASΔN mRNA in various combinations. As described in Example 2, cGASΔN mRNA and 22:0 LPC were formulated in LNPs either separately or in combination.

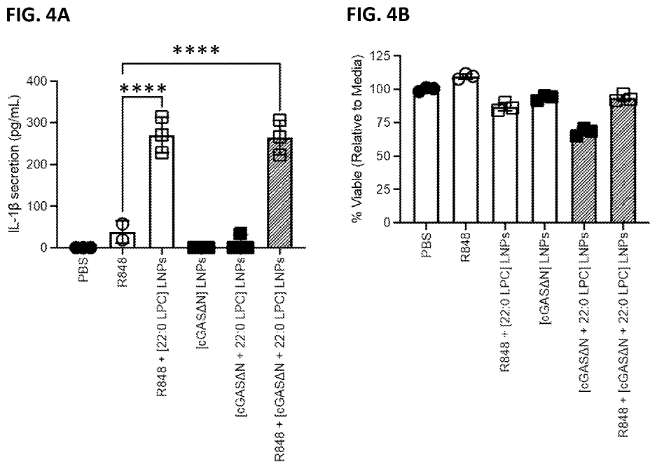

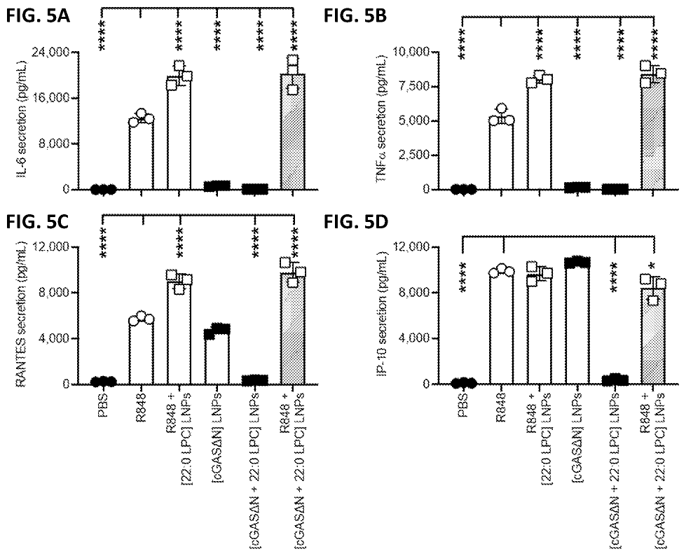

Flt3L BMDCs treated with R848 and [cGASΔN+22:0 LPC] LNPs can be hyperactivated. DCs were assessed for their potential to be hyperactivated in response to treatment with R848+[cGASΔN+22:0 LPC] LNPs; hyperactivation was typified by the DCs' ability to secrete IL-1β while remaining viable. DCs treated with PBS (unstimulated) or treated with only R848 produced minimal or no IL-1β. As expected, combining R848 and 22:0 LPC treatments resulted in IL-1β production 48 hours later (FIG. 4A). Treatment of cells using cGASΔN mRNA-containing LNPs did not induce IL-1β. cGASΔN treatment combined with 22:0 LPC also did not result in IL-1β release (FIG. 4A). cGAS/STING signaling is likely insufficient in producing the pro-form of IL-1β protein that is required for the 22:0 LPC-mediated release of mature IL-1β. However, combining all three stimuli, R848, cGASΔN, and 22:0 LPC resulted in the release of IL-1β (FIG. 4A). This result demonstrated that cGASΔN treatment did not inhibit hyperactivation. Next, cell viability was assessed using an LDH release assay to confirm the cells were hyperactivating and not pyroptosing. Across the treatment conditions tested, the DCs had a similar viability profile to unstimulated cells, confirming that the DCs were hyperactivating (FIG. 4B) Together, these data suggest that DCs can be simultaneously treated with R848, 22:0 LPC, and cGASΔN mRNA and hyperactivate.

[cGASΔN and 22:0 LPC] LNP+R848 treatment activates NF-kB signaling and cGAS-STING pathways in Flt3L BMDCs. To further confirm the activity of the individual stimuli, IL-6 and TNFα production was quantified. These two genes are primarily regulated by NF-kB and thus by R848 stimulation. When R848 was added as a treatment, IL-6 and TNFα were produced (FIG. 5A-5B). Adding to R848 treatment with LNPs containing 22:0 LPC increased both IL-6 and TNFα secretion. cGASΔN alone did not stimulate IL-6 and TNFα secretion (FIG. 5A-5B). cGAS/STING signaling primarily activates IRF3 and not NF-kB. In addition, secretion of RANTES and IP-10, which are known to be regulated by cGAS/STING signaling, were measured. Cells treated with PBS (unstimulated) did not express RANTES or IP-10. In contrast, cGASΔN stimulation induced the expression of RANTES and IP-10, confirming that our mRNA transcript encoded an active cGAS that was successfully delivered to cells and expressed (FIG. 5C-5D). Unexpectedly, when cGASΔN was delivered together with 22:0 LPC, RANTES and IP-10 expression were inhibited compared to single cGASΔN treatment. When cells were stimulated with R848 alone or in combination with 22:0 LPC in a hyperactivation, cells produced RANTES and IP-10 with no 22:0 LPC-mediated inhibition observed. Despite the surprising inhibition observed when cGASΔN was combined with 22:0 LPC, combining all three resulted in the expression of RANTES and IP-10 (FIG. 5C-5D).

Altogether, the treatment of murine DCs with the various combinations of R848, 22:0 LPC, and cGASΔN demonstrated several things. Firstly, cGASΔN mRNA was successfully formulated in LNPs for delivery into DCs and was translated into a protein. cGASΔN protein was active, as indicated by the treatment's ability to induce RANTES and IP-10 production. Secondly, in combination with hyperactivating stimuli, cGASΔN treatment did not reduce secretion of the key hyperactivation cytokine IL-1β. Finally, when cells were stimulated with all three stimuli, inflammatory genes regulated by one stimulus were not impaired by the effects of the other two stimuli. These data demonstrate that cGASΔN treatment can be combined with hyperactivating stimuli to instruct DCs to produce the inflammatory milieu of both processes simultaneously. This combination leads to a unique DC activation state which can have potential advantages in immune responses.

### Example 4: Treatment of Human Dendritic Cells with cGASΔN and Hyperactivating Stimuli Results in Activation of NLRP3 Inflammasome and cGAS-STING Pathways

### Materials & Methods

Human monocyte-derived dendritic cell (moDC) generation. Human monocytes were isolated from Leukopaks purchased from Miltenyi Inc. (San Jose, CA) using the StraightFrom Leukopak CD14 microbead kit according to the manufacturer's instructions. Monocytes were then aliquoted and frozen in fetal bovine serum containing 10% dimethyl sulfoxide. For studies with monocyte-derived dendritic cell (moDC) cultures, monocytes were thawed and cultured in RPMI medium containing 10% FBS, 50 units/mL penicillin, 50 mg/mL streptomycin, 2 mM L-glutamine, 1 mM sodium pyruvate, 50 mM beta-mercaptoethanol, 10 mM HEPES, and Gibco MEM non-essential amino acids (R10 media). To differentiate monocytes into moDCs, recombinant human GM-CSF (50 ng/mL) and IL-4 (25 ng/mL) were added to R10 media. Cells were cultured for 6 days with GM-CSF and IL-4, with an additional cell feeding with R10 media containing GM-CSF and IL-4 on day 3. On day 6 of differentiation, moDC are collected for use and a portion are stained to confirm successful differentiation. Routinely, >80% of live cells are CD11c+CD209+ by flow cytometry using a BD Symphony A3.

Hyperactivation of moDC and cGASΔN delivery. Six days following differentiation from monocytes, moDC were collected and counted. Cells were plated at 100,000 cells/well in 96-well plates. moDC were treated with 1 ug/mL R848 and LNPs containing 22:0 LPC and/or cGASΔN mRNA. Forty-eight hours after stimulation, cell culture supernatant was collected. Cytokine expression was measured using the LEGENDplex Human Anti-viral Response Panel (Biolegend) according to manufacters' instructions. Cell viability was assessed by measuring LDH release into fresh supernatant using the CyQUANT LDH Cytotoxicity Assay (Invitrogen) according to manufacturer's instructions. Cells were collected and stained to measure expression of DC molecules involved in T cell activation such as CD40, CD80, CD83, CD86, CCR7, HLA-ABC, and HLA-DR expression was assessed by flow cytometry using a BD Symphony A3.

### Results

We hypothesized that combining cGASΔN-mediated STING signaling with hyperactivation could help build immunotherapies and vaccines that could provide better protection than currently available options. Thus, we tested combining cGASΔN therapy with hyperactivation on human DCs. As a source of DCs, human monocytes were differentiated into DCs by culturing in R10 media containing recombinant GM-CSF and IL-4 for six days. Cells were confirmed to be DCs by CD11c and CD209 staining on day 6, and cells were used for treatment with our three stimuli of interest: R848, 22:0 LPC and cGASΔN.

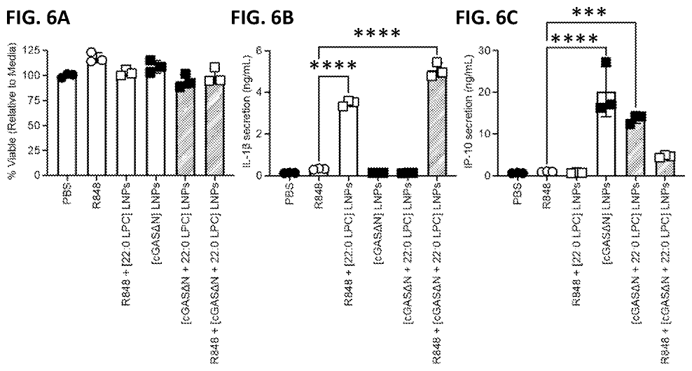

moDCs treated with R848 and [cGASΔN+22:0 LPC] LNPs are hyperactivated and allow for cGAS-STING pathway activation. We first started by confirming major conclusions obtained using murine DCs. Combinations of these three treatments (R848, 22:0 LPC, and cGASΔN) had a similar impact on human DC viability as compared to DCs treated with PBS (unstimulated) (FIG. 6A). When IL-1β release was measured, we observed that treatment with PBS (no stimulation) or R848 produced minimal IL-1β (FIG. 6B). As expected, the combination of R848 and 22:0 LPC delivered via LNP induced IL-1β secretion. Similar to mice, cGASΔN treatment alone or in combination with 22:0 LPC did not induce secretion of IL-1β (FIG. 6B). When cGASΔN was combined with the hyperactivating combination of R848 and 22:0 LPC, IL-1β secretion was maintained or increased (FIG. 6B). IP-10 secretion was quantified to ensure that cGASΔN mRNA formulated in LNPs could induce signaling. cGASΔN treatment alone induced IP-10 expression. In contrast to the murine data, addition of 22:0 LPC to cGASΔN LNPs did not inhibit IP-10 expression (FIG. 6C). Also, in contrast to the murine data, R848 treatment (either alone or in combination with 22:0 LPC) was insufficient in inducing IP-10 expression (FIG. 6C). When all three stimuli were combined, cGASΔN, R848, and 22:0 LPC were able to induce IP-10 expression (FIG. 6C). For the donor shown, a reduction in IP-10 was observed when all three stimuli were combined compared to cGASΔN treatment alone. However, this reduction has not been consistently observed in the 4 donors tested. Regardless of this reduction, the combination of cGASΔN, R848, and 22:0 LPC resulted in IP-10 expression that was significantly higher than what was detected in the unstimulated condition (FIG. 6C). Altogether these results suggested that like in murine DCs, human DCs are capable of mediating cGAS/STING activation while also being hyperactivated.

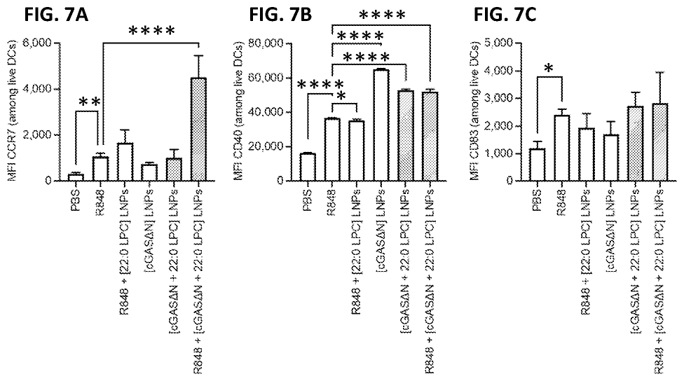

moDCs treated with R848 and [cGASΔN+22:0 LPC] LNPs upregulate surface proteins required for T cell activation. Beyond the production of a specific cytokine milieu, DCs need to express cell surface proteins that are important in the activation of T cells. One such protein is the chemokine receptor CCR7 that is required for DC trafficking from peripheral tissues to the lymph node where they can prime T cells. DCs were stained for CCR7 expression after being treated with various combinations of R848, 22:0 LPC and cGASΔN. As observed previously, treatment with R848 alone increased CCR7 expression, and hyperactivation treatment with R848 in combination with 22:0 LPC led to a further increase in CCR7 (FIG. 7A). cGASΔN treatment also increased CCR7 expression compared to unstimulated cells, and addition of 22:0 LPC did not have a significant effect on CCR7 expression (FIG. 7A). Interestingly, combining cGASΔN treatment with R848 and 22:0 LPC resulted in the largest shift in CCR7 expression (FIG. 7A). Importantly, combining cGASΔN with hyperactivation did not inhibit CCR7 expression, and the increased CCR7 expression could enable more efficient migration of DCs to lymph nodes.

We proceeded to examine other key cell surface proteins on dendritic cells that are important to their function. CD40 is a receptor that engages CD40L on CD4 T cells and ultimately enhances T cell responses. CD83 is also involved in T cell activation and is commonly used as an indicator of DC activation. Compared to unstimulated cells, CD40 expression was upregulated when DCs were treated with R848 with or without 22:0 LPC, and a further enhancement was observed when cells were treated with cGASΔN with or without 22:0 LPC (FIG. 7B). Combining cGAS signaling with hyperactivation also led to an increase in CD40 expression compared to unstimulated cells (FIG. 7B). CD83 expression was more variable compared to CD40 staining, but again combining cGASΔN treatment with hyperactivation treatment did not eliminate the expression of CD83 (FIG. 7C).

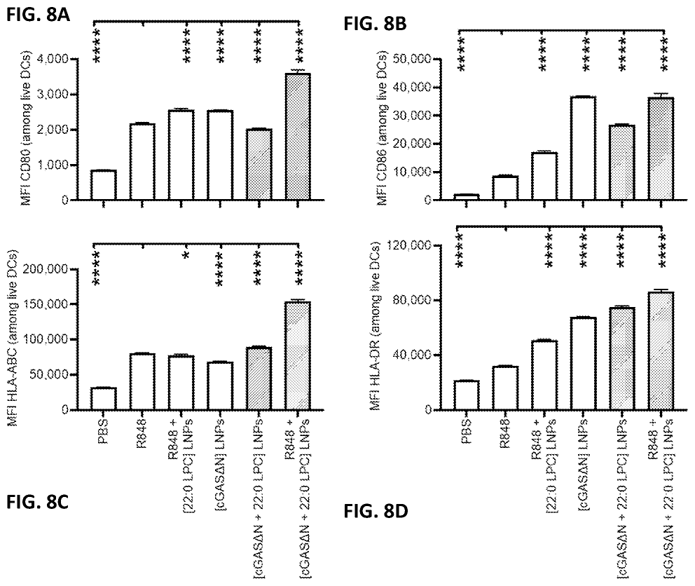

CD80 and CD86 are costimulatory molecules that engage T cells during antigen presentation and serve as a confirmatory signal that a presented non-self antigen is indeed dangerous. When DCs are activated with R848 or cGASΔN or when DCs are hyperactivated with R848 and 22:0 LPC, CD80 and CD86 expression increases (FIG. 8A-8B). When cGASΔN and hyperactivating treatments are combined, DCs continue to upregulate CD80 and CD86 compared to unstimulated cells (FIG. 8A-8B). Thus, when both cellular processes are engaged, DCs are still able to supply T cells with co-stimulatory signals.

Finally, DCs were stained for HLA-ABC and HLA-DR which are Class I and Class II MHC molecules that present antigens to CD8 and CD4 T cells, respectively. Compared to unstimulated cells, R848 treatment with or without the addition of 22:0 LPC increased the expression of HLA-ABC (FIG. 8C). Treatment of DCs with cGASΔN with or without 22:0 LPC also had a similar effect on HLA-ABC expression (FIG. 8C). Surprisingly, when combining cGASΔN treatment and hyperactivation, a significant increase in HLA-ABC was observed compared to the other stimuli combinations. When HLA-DR staining was analyzed, R848 treatment was observed to increase HLA-DR expression compared to unstimulated cells (FIG. 8D). A further increase was then observed when hyperactivating with the combination of R848 and 22:0 LPC (FIG. 8D). cGASΔN treatment was more effective than R848 treatment alone or as part of a hyperactivation (FIG. 8D). 22:0 LPC addition to cGASΔN treatment further enhanced HLA-DR expression compared to cGASΔN treatment alone (FIG. 8D). Finally, combining cGASΔN treatment with hyperactivation led to the highest HLA-DR expression of all the experimental conditions (FIG. 8D). Combining all three stimuli resulted in the highest expression of MHC Class I and Class II molecules, which can enable more effective antigen presentation to T cells.

Studies using human DCs suggest that they generally react similarly to cGASΔN, R848, and 22:0 LPC compared to mouse DCs. One unexpected difference was when cells were treated with cGASΔN with or without 22:0 LPC. While mouse DCs showed an inhibition of IP-10 when 22:0 LPC was added, human DCs did not have the same result. More importantly, combining cGASΔN, R848, and 22:0 LPC did not prevent human DCs from hyperactivating and producing cytokines such as IL-1β and IP-10. Further analysis of human DCs for cell surface expression of key proteins involved in T cell activation demonstrated that the combination of the three stimuli did not inhibit expression. These proteins were still inducible and in some cases such as HLA-ABC and HLA-DR, expression was further increased compared to cGASΔN treatment alone or hyperactivation. Altogether, these data collected from treatment of human DCs suggest that cGASΔN treatment can be combined with hyperactivation to induce a cell state where the two processes complement each other and can potentially have additive effects on cellular function for the induction of adaptive immune responses.

### Example 5: Combining cGASΔN mRNA and Hyperactivation as a Vaccination Strategy to Improve Immune Responses

By utilizing mRNA encoding cGASΔN, DCs can be activated to produce chemical signals that promote T cell effector responses. Hyperactivation of DCs produces a complementary set of chemical signals. In particular, hyperactivation induces pro-inflammatory cytokines and add to their cytokine repertoire IL-1β, a critical cytokine for memory T cell formation. By combining stimuli to engage cGAS/STING signaling and hyperactivation, a broader set of inflammatory signals can be produced to maximize the immune response initiated by DCs.

To test this model, vaccination strategies that utilize various combinations of cGASΔN, R848, and 22:0 LPC as adjuvants are compared in vivo. The goal of vaccination is to direct immune responses against an antigen of interest or a complex source of antigens of interest (e.g., model antigen, tumor-associated antigen, neoantigen, microbial-derived antigens, etc.). Immune responses are easily detected using well-established immunological methods and common reagents (e.g., tetramer staining, ELISpot assay, ELISA, flow cytometry, etc.). An immunogenic composition is prepared, for instance, by loading an mRNA transcript encoding an antigen of interest into lipid nanoparticles (LNPs). These LNPs are suitable for administration to mammalian subjects to achieve expression of the antigen of interest in vivo for initiation of an adaptive immune response.

The aim of this experiment is to test whether cGASΔN, delivered as mRNA in an LNP, can be used as a vaccine adjuvant to improve immune responses to a model antigen. cGASΔN, R848, and 22:0 LPC are contemplated to improve DC function by enhancing de novo T cell activation and memory T cell reactivation. These improvements can have a positive downstream effect on effector and memory T cell responses. Improved T cell activation can also have further positive downstream effects on B cells when B cells undergo germinal center reactions that require T cell engagement.

TABLE 5-1
Immunization Strategy

| | | | Innate | Effector | Memory |
|---|---|---|---|---|---|
| Group | Antigen | DC Stimuli | Pathway | Response | Response |
| 1 | None | [GFP] Control LNPs | None | − | − |
| 2 | OVA | [GFP] Control LNPs | None | + | − |
| 3 | OVA | R848 + | NFkB | + | − |
| | | [GFP] Control LNPs | | | |
| 4 | OVA | R848 + | NFkB | ++ | ++ |
| | | [22:0 LPC] LNPs | NLRP3 | | |
| 5 | OVA | [cGASΔN] LNPs | IRF3 | +++ | + |
| 6 | OVA | [cGASΔN + 22:0 LPC] LNPs | IRF3 | +++ | + |
| 7 | OVA | R848 + | NFkB | ++++ | +++ |
| | | [cGASΔN] LNPs | NLRP3 | | |
| | | | IRF3 | | |
| 8 | OVA | R848 + | NFkB | +++++ | ++++ |
| | | [cGASΔN + 22:0 LPC] LNPs | NLRP3 | | |
| | | | IRF3 | | |

### Methods

In vivo immunization with [cGASΔN+22:0 LPC] LNPs plus mRNA antigen in LNPs. C57BL/6J mice are immunized subcutaneously with LNPs as detailed in Table 5-1. OVA mRNA-loaded LNPs are used to deliver transcripts for production of OVA antigen in vivo, in combination with LNPs containing either adjuvant cGASΔN mRNA or non-adjuvanting GFP mRNA. The OVA mRNA dose is fixed at 5 pig/mouse, while the adjuvant cGASΔN mRNA is dosed at about 1.5 pig/mouse and 22:0 LPC is dosed at about 60 gig/mouse (Table 5-1). Mice are given a primary immunization on Day 0, with a boost immunization of the same doses on Day 7. For assessment of short-term effector responses, on Day 14, blood and secondary lymphoid organs are collected. For assessment of long-term memory responses, on Day 40, blood and secondary lymphoid organs are collected. Blood is collected for measurement of antibody and T cell responses. Serum is collected from the blood using serum separation tubes, while blood for cellular analysis is collected using K2EDTA tubes. After blood collection, mice are euthanized, and the draining lymph nodes and spleen are collected and processed into single cell suspensions. Expected responses are shown in Table 5-1.

OVA-specific T cell tetramer assessment. OVA-specific T cells in the blood and draining lymph node of mice receiving OVA LNP immunization are assessed 14 and 40 days post primary immunization. CD8+ T cells specific for SIINFEKL (SEQ ID NO:17), an MHC-I-restricted T cell epitope of ovalbumin (OVA), are quantified in the blood and dLN using by SIINFEKL-tetramer staining. Briefly, red blood cells are lysed using a RBC lysis buffer, with lysis completed twice to remove all RBCs in the blood. Cells are washed, then stained for viability (Live/Dead), the SIINFEKL-tetramer binding (MBL), and CD3, CD4, and CD8 expression. Cells are fixed with 4% paraformaldehyde, and counting beads are added before running to permit total cell counts to be determined. Data are collected using a BD FACS Symphony and analyzed using Flowjo (BD).

Effector and memory T cell measurement. The frequency of T effector and T memory cells are assessed by flow cytometry, in the blood and draining lymph node of mice receiving OVA LNP immunization. Briefly cell suspensions are stained with CD3, CD4, CD8, CD62L and CD44 antibodies to measure the frequency of T effector cells (CD44^{Low}CD62L^{neg}) and T memory cells (CD44^{high}CD62L⁺) using a BD FACS Symphony instrument and data is analyzed using Flowjo (BD).

OVA-specific antibody assessment. OVA-specific antibodies in the serum of mice receiving OVA LNP immunization are assessed 14 and 40 days post primary immunization. OVA-specific total IgG, IgG1, and IgG2b are assessed by ELISA. Briefly, ELISA plates are coated with 10 μg/mL Endofit Ovalbumin (Invivogen) overnight, then washed and blocked with 2% bovine serum albumin. Plates are washed again, and then serum is added to the plates at a 1:500 dilution, followed by 1:5 dilutions for a total of 7 serum dilutions. Samples are washed, then incubated with detection antibodies specific for IgG, IgG1, or IgG2b, which are conjugated to HRP (Southern Biotech), to detect total, Th2-skewed, and Th1-skewed OVA-specific antibodies respectively. Plates are washed, then incubated with TMB, and stop solution is added once color development is complete.

OVA-specific T cell responses. OVA-specific T cell responses are determined from secondary lymphoid organ activities. Post-immunization, draining lymph nodes and spleens are collected from the mice at the early (Day 14) and late (Day 40) time points. Harvested lymph nodes and spleens are dissociated into single cell suspensions, which are used in ELISPOT assays. ELISPOT is used to detect IFN7 and IL-5 secretion by T cells, which are indicative of Th1 and Th2 responses, respectively. Cells from draining lymph nodes and spleens are plated in RPMI medium containing 10% FBS, 50 units/mL penicillin, 50 mg/mL streptomycin, 2 mM L-glutamine, 1 mM sodium pyruvate, 50 mM beta-mercaptoethanol, 10 mM HEPES, and Gibco MEM non-essential amino acids (R10 media). Cells are plated at 200,000 cells/well in a 96 well ELISPOT plate and restimulated with 10 mcg/mL OVA peptivator or 1 mcg/mL OVA peptide antigens. As controls, additional plated cells are left unstimulated or stimulated with irrelevant antigens (not used in vaccination). Completion of the assay results in spots that can be visually quantified where cytokines were secreted by T cells, as a method to quantify the number of T cells responding to restimulation.

### Results

Using the assays described above to assess short term (2 weeks post vaccination) and long term (>4 weeks post vaccination) adaptive T cell immunity, effector and memory responses are analyzed. Table 5-1 lists experimental groups to be included in the study. As a negative control, mice of Group 1 receive a sham injection containing no antigen and no adjuvants to serve as a baseline where little to no antigen-specific immune responses are expected to be elicited. Group 2 mice receive mRNA encoding a model antigen (OVA) formulated in LNPs, which represents a standard LNP vaccination protocol. The remaining groups all receive LNP-loaded with mRNA encoding OVA in combination with adjuvants. Depending upon the type of stimuli administered, one or more of the following are expected: i) activate cells (induce NFkB signaling); ii) hyperactivate DCs (induce NLRP3 pathway activation); and iii) induce IRF3 signaling in DCs. For instance, Group 4 mice receive R848+22:0 LPC, which is expected to hyperactivate DCs that activate the NLRP3 pathway in addition to the NFkB pathway leading to conventional pro-inflammatory cytokine secretion (IL-6 and TNFα) and IL-1β production. Group 5 mice receive LNPs that encode cGASΔN, which is expected to induce a Type I IFN response via IRF3. Mice of Group 6 and Group 7 receive two out of the three stimuli, while mice of Group 8 receive all three stimuli (R848, 22:0 LPC, and cGASΔN).

cGASΔN expression in vivo is contemplated to stimulate DC signals that are particularly beneficial to the immediate effector immune responses. Hyperactivating conditions are also contemplated to benefit effector responses, albeit in the absence of Type I IFN signaling. Additionally, hyperactivation-induced IL-1β signaling leads to improved memory formation. Thus, by combining cGAS signaling and hyperactivation, a very strong effector response and a durable memory response are contemplated to be elicited. By immunizing with a sham treatment (Group 1), little to no effector and memory responses are likely to be observed and immunizing with antigen mRNA alone or in combination with R848 (Groups 2 and 3) is contemplated to lead to a small effector response and minimal memory formation. Group 4 treatment hyperactivates DCs and is expected to be an improvement over Group 2 treatment. In comparison, Group 5 and Group 6 treatments engage only cGAS signaling in DCs and are therefore contemplated to lead to strong effector responses, but less durable memory responses than Group 4 treatment. Group 7 is a hyperactivating treatment condition. Group 7 and Group 8 are contemplated to yield the best effector and memory responses given that they engage both cGAS and hyperactivation pathways. Since hyperactivating lipids engage cellular processes beyond NLRP3 activation, the Group 8 treatment, which includes 22:0 LPC, is contemplated to result in superior effector and memory responses.

The three adjuvanting stimuli can be formulated in various ways. For example, cGASΔN mRNA can be encapsulated in LNPs alone or in combination with 22:0 LPC. R848 can be administered as an individual component, within an LNP, or within an LNP containing one or both of the other stimuli. In addition, the chronology of administration of stimuli can be varied. For example, it may be beneficial to administer the antigen mRNA-loaded LNPs in combination with cGASΔN mRNA-loaded LNPs and R848 in a prime injection to provide a strong effector signal via type I IFN, followed by a boost injection of the antigen mRNA-loaded LNPs in combination with 22:0 LPC-loaded LNPs and R848 to provide the memory signal necessary to generate durable immunity.

Studying T and B cell responses to vaccination generates in vivo data that is contemplated to demonstrate that combining hyperactivation with cGAS signaling will result in improvements in antigen-specific immune responses. Assessment of how administration of various DC stimuli affects B cell responses is accomplished by measuring total antigen-specific IgG antibodies, as well as antibody isotypes such as IgG1 (associated with TH2 responses) and IgG2b (associated with TH1 responses) in the serum of immunized mice. Based on previous studies using cGASΔN LNPs, we expect that Mice immunized with LNPs loaded with mRNA encoding an antigen and mRNA encoding cGASΔN is expected to result in TH1-skewed responses.

We claim:

1. A composition comprising an mRNA encapsulated in a lipid nanoparticle (LNP), wherein the mRNA comprises a coding region of a constitutively active cyclic GMP-AMP synthase (cGAS), and the LNP comprises a first phospholipid, and at least one lipid selected from the group consisting of an ionizable lipid, a pegylated lipid, a structural lipid, a second phospholipid, and mixtures thereof, wherein the first phospholipid comprises a lysophosphatidylcholine (LPC) with a single C13-C24 acyl chain.

2. A composition comprising a first mRNA and a second mRNA encapsulated in a lipid nanoparticle (LNP), wherein the first mRNA comprises a coding region of a constitutively active cyclic GMP-AMP synthase (cGAS), the second mRNA comprises a coding region of an antigen; and the LNP comprises a first phospholipid, and at least one lipid selected from the group consisting of an ionizable lipid, a pegylated lipid, a structural lipid, a second phospholipid, and mixtures thereof, wherein the first phospholipid comprises a lysophosphatidylcholine (LPC) with a single C13-C24 acyl chain.

3. A composition comprising an mRNA encapsulated in a lipid nanoparticle (LNP), wherein the mRNA comprises a first coding region and a second coding region separated by a 2A-like sequence, wherein the first coding region is a coding region of a constitutively active cyclic GMP-AMP synthase (cGAS) and the second coding region is a coding region of an antigen or the first coding region is a coding region of an antigen and the second coding region is a coding region of a constitutively active cyclic GMP-AMP synthase (cGAS), and the LNP comprises a first phospholipid, and at least one lipid selected from the group consisting of an ionizable lipid, a pegylated lipid, a structural lipid, a second phospholipid, and mixtures thereof, wherein the first phospholipid comprises a lysophosphatidylcholine (LPC) with a single C13-C24 acyl chain.

4. The composition of any one of claims 1-3, wherein the at least one lipid comprises an ionizable lipid, a second phospholipid, a pegylated lipid, and a structural lipid.

5. The composition of any one of claims 1-4, wherein the ionizable lipid comprises:
i) 8-[(2-hydroxyethyl)[6-oxo-6-(undecyloxy)hexyl]amino]-octanoic acid, 1-octylnonyl ester (SM-102) or analogs or derivatives thereof, and/or
ii) 6-((2-hexyldecanoyl)oxy)-N-(6-((2-hexyldecanoyl)oxy)hexyl)-N-(4-hydroxybutyl)hexan-1-aminium (ALC-0315) or analogs or derivatives thereof, and/or
iii) (6Z,9Z,28Z,31Z)-heptatriaconta-6,9,28,31-tetraen-19-yl 4-(dimethylamino)butanoate (DLin-MC3-DMA) or analogs or derivatives thereof.

6. The composition of any one of claims 1-5, wherein the pegylated lipid is selected from the group consisting of a PEG-modified phosphatidyiethanolamine, a PEG-modified phosphatide acid, a PEG-modified ceramide, a PEG-modified dialkylamine, a PEG-modified diacylglycerol, a PEG-modified dialkylglyerol, and combinations thereof.

7. The composition of any one of claims 1-5, wherein the pegylated lipid comprises polyethylene glycol [PEG]2000 dimyristoyl glycerol [DMG].

8. The composition of any one of claims 1-7, wherein the structural lipid is selected from the group consisting of cholesterol, fecosterol, sitosterol, ergosterol, campesterol, stigmasterol, brassicasterol, tomatidine, ursolic acid, alpha-tocopherol, and combinations thereof.

9. The composition of any one of claims 1-7, wherein the structural lipid comprises cholesterol.

10. The composition of any one of claims 1-9, wherein the second phospholipid comprises:
i) a hydrophilic head moiety selected from the group consisting of phosphatidyl choline, phosphatidyl ethanolamine, phosphatidyl glycerol, phosphatidyl serine, phosphatidic acid, 2-lysophosphatidyl choline, and sphingomyelin; and
ii) one or more fatty acid tail moieties selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid.

11. The composition of any one of claims 1-9, wherein the second phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1-palmitoyl-2-oleoyl-sn-glycero-3-phosphocholine (POPC),
1,2-di-O-octadecenyl-sn-glycero-3-phosphocholine,
1-oleoyl-2-cholesterylhemisuccinoyl-sn-glycero-3-phosphocholine,
1,2-dilinolenoyl-sn-glycero-3-phosphocholine,
1,2-diarachidonoyl-sn-glycero-3-phosphocholine,
1,2-didocosahexaenoyl-sn-glycero-3-phosphocholine,
1,2-dioleoyl-sn-glycero-3-phosphoethanola mine (DOPE),
1,2-diphytanoyl-sn-glycero-3-phosphoethanolamine,
1,2-distearoyl-sn-glycero-3-phosphoethanolamine,
1,2-dilinoleoyl-sn-glycero-3-phosphoethanolamine,
1,2-dilinolenoyl-sn-glycero-3-phosphoethanolamine,
1,2-diarachidonoyl-sn-glycero-3-phosphoethanolamine,
1,2-didocosahexaenoyl-sn-glycero-3-phosphoethanolamine,
1,2-dioleoyl-sn-glycero-3-phospho-rac-(1-glycerol) sodium salt (DOPG),
sphingomyelin, and
combinations thereof.

12. The composition of claim 11, wherein the second phospholipid comprises 1,2-distearoyl-sn-glycero-3-phosphocholine (DSPC).

13. The composition of any one of claims 1-12, wherein the composition further comprises at least one excipient.

14. The composition of claim 13, wherein the excipient comprises sucrose.

15. A composition comprising:
i) an mRNA complexed with one or more lipids (RNA-Lipoplex); and
ii) a lysophosphatidylcholine (LPC) with a single C13-C24 acyl chain, wherein the mRNA comprises a coding region of a constitutively active cyclic GMP-AMP synthase (cGAS), and the one or more lipids comprise a first lipid and a second lipid.

16. A composition comprising:
i) a first mRNA and a second mRNA complexed with one or more lipids (RNA-Lipoplex); and
ii) a lysophosphatidylcholine (LPC) with a single C13-C24 acyl chain;
wherein the first mRNA comprises a coding region of a constitutively active cyclic GMP-AMP synthase (cGAS), and the second mRNA comprises a coding region of an antigen, and the one or more lipids comprise a first lipid and a second lipid.

17. A composition comprising:
i) an mRNA complexed with one or more lipids (RNA-Lipoplex); and
ii) a lysophosphatidylcholine (LPC) with a single C13-C24 acyl chain,
wherein the mRNA comprises a first coding region and a second coding region separated by a 2A-like sequence, the first coding region is a coding region of a constitutively active cyclic GMP-AMP synthase (cGAS) and the second coding region is a coding region of an antigen or the first coding region is a coding region of an antigen and the second coding region is a coding region of a constitutively active cyclic GMP-AMP synthase (cGAS), and the one or more lipids comprise a first lipid and a second lipid.

18. The composition of any one of claims 15-17, wherein the first lipid is a cationic lipid, and the second lipid is a neutral or anionic lipid.

19. The composition of claim 18, wherein the cationic lipid comprises one or both of:
i) 1,2-di-O-octadecenyl-3-trimethylammonium propane (DOTMA) or analogs or derivatives thereof, and
ii) 1,2-dioleoyl-3-trimethylammonium propane (DOTAP) or analogs or derivatives thereof.

20. The composition of claim 18 or claim 19, wherein the neutral or anionic lipid comprises:
i) 1,2-di-(9Z-octadecenoyl)-sn-glycero-3-phosphoethanolamine (DOPE) or analogs or derivatives thereof, and/or
ii) cholesterol or analogs or derivatives thereof; and/or
iii) 1,2-dioleoyl-sn-glycero-3-phosphocholine (DOPC) or analogs or derivatives thereof.

21. The composition of any one of claims 1-20, wherein the acyl chain of the LPC is a C21-C24 acyl chain.

22. The composition of any one of claims 1-20, wherein the acyl chain of the LPC is a C22 acyl chain.

23. The composition of any one of claims 1-22, wherein the acyl chain of the LPC is fully saturated.

24. The composition of claim 23, wherein the LPC comprises 1-behenoyl-2-hydroxy-sn-glycero-3-phosphocholine [LPC(22:0)].

25. The composition of any one of claims 1-24, further comprising a TLR7/8 agonist.

26. The composition of claim 25, wherein the TLR7/8 agonist is a small molecule with a molecule weight of 900 daltons or less.

27. The composition of claim 26, wherein the TLR7/8 agonist comprises an imidazoquinoline compound.

28. The composition of claim 27, wherein the TLR7/8 agonist comprises resiquimod (R848).

29. The composition of any one of claims 25-28, wherein the LPC comprises LPC(22:0), and the TLR7/8 agonist comprises resiquimod (R848).

30. The composition of any one of claims 1-29, wherein the mRNA or the first mRNA and the second mRNA comprises a 5′ untranslated region (5′UTR) and a 3′ untranslated region (3′UTR).

31. The composition of any one of claims 1-30, wherein the mRNA comprises a 5′ cap structure.

32. The composition of any one of claims 1-31, wherein the mRNA comprises a polyA tail.

33. The composition of any one of claims 1-32, wherein the mRNA is a nucleoside-modified mRNA.

34. A nucleic acid comprising: i) a coding region an antigen, and ii) a coding region of a constitutively-active cyclic GMP-AMP synthase (cGAS), optionally wherein the nucleic acid is mRNA, optionally wherein the nucleic acid is DNA.

35. An expression vector comprising the nucleic acid of claim 34.

36. The composition of any one of claims 1-35, wherein the constitutively-active cGAS has a greater propensity to self DNA reactivity than its wild-type counterpart.

37. The composition of any one of claims 1-36, wherein the cGAS is a truncated cGAS devoid of an amino-terminal phosphoinositide-binding domain (cGASΔN).

38. The composition of claim 37, wherein the cGASΔN comprises the amino acid sequence of SEQ ID NO:1 or the amino acid sequence at least 85%, 90%, 91%, 92%, 93%, 94%, 95%, 96%, 97%, 98%, or 99% identical to SEQ ID NO:1.

39. The composition of claim 38, wherein the cGASΔN comprises:
(i) the amino acid sequence of SEQ ID NO:2, SEQ ID NO:3, SEQ ID NO:4, SEQ ID NO:5, SEQ ID NO:6, SEQ ID NO:7 or SEQ ID NO:8, or the amino acid sequence at least 95%, 96%, 97%, 98% or 99% identical to SEQ ID NO:2, SEQ ID NO:3, SEQ ID NO:4, SEQ ID NO:5, SEQ ID NO:6, SEQ ID NO:7 or SEQ ID NO:8; or
(ii) the consensus amino acid sequence of SEQ ID NO:9.

40. The composition of claim 38, wherein the cGASΔN is encoded by the nucleotide sequence of SEQ ID NO:17.

41. The composition of any one of claims 37-40, wherein the coding region of the cGASΔN is in operable combination with a start codon (ATG).

42. The composition of any one of claims 1-41, wherein the antigen is a tumor antigen.

43. The composition of claim 42, wherein the tumor antigen is a tumor-associated antigen.

44. The composition of claim 42, wherein the tumor antigen is a neoantigen.

45. The composition of any one of claims 1-41, wherein the antigen comprises a microbial antigen.

46. The composition of claim 44, wherein the microbial antigen comprises a viral antigen, a bacterial antigen, a protozoan antigen, or a fungal antigen.

47. The composition of any one of claims 1-41, wherein the antigen comprises a surface protein or fragment thereof of a pathogen.

48. The composition of claim 47, wherein the pathogen is capable of causing disease in human subjects.

49. The composition of claim 47 or claim 48, wherein the pathogen is a virus.

50. The composition of claim 49, wherein the virus is a SARS-CoV-2.

51. The composition of claim 50, wherein the antigen is a spike (S) glycoprotein of the SARS-CoV-2, optionally wherein the spike glycoprotein is a pre-fusion stabilized variant.

52. The composition of any one of claims 1-51, wherein the composition does not comprise lipopolysaccharide (LPS) or monophosphoryl lipid A (MPLA).

53. The composition of any one of claims 1-52, wherein the composition does not comprise oxidized 1-palmitoyl-2-arachidonoyl-sn-glycero-3-phosphorylcholine (oxPAPC) or a species of oxPAPC.

54. The composition of claim 53, wherein the composition does not comprise 2-[[(2R)-2-[(E)-7-carboxy-5-hydroxyhept-6-enoyl]oxy-3-hexadecanoyloxypropoxy]-hydroxyphosphoryl]oxyethyl-trimethylazanium(HOdiA-PC), [(2R)-2-[(E)-7-carboxy-5-oxohept-6-enoyl]oxy-3-hexadecanoyloxypropyl]2-(trimethylazaniumyl)ethyl phosphate (KOdiA-PC), 1-palmitoyl-2-(5-hydroxy-8-oxo-octenoyl)-sn-glycero-3-phosphorylcholine (HOOA-PC), 2-[[(2R)-2-[(E)-5,8-dioxooct-6-enoyl]oxy-3-hexadecanoyloxypropoxy]-hydroxyphosphoryl]oxyethyl-trimethylazanium (KOOA-PC), [(2R)-3-hexadecanoyloxy-2-(5-oxopentanoyloxy)propyl]2-(trimethylazaniumyl)ethyl phosphate (POVPC), [(2R)-2-(4-carboxybutanoyloxy)-3-hexadecanoyloxy propyl]2-(trimethylazaniumyl)ethyl phosphate (PGPC), [(2R)-3-hexadecanoyloxy-2-[4-[3-[(E)-[2-[(Z)-oct-2-enyl]-5-oxocyclopent-3-en-1-ylidene]methyl]oxiran-2-yl]butanoyloxy]propyl]2-(trimethylazaniumyl)ethyl phosphate (PECPC), [(2R)-3-hexadecanoyloxy-2-[4-[3-[(E)-[3-hydroxy-2-[(Z)-oct-2-enyl]-5-oxocyclopentylidene]methyl]oxiran-2-yl]butanoyloxy]propyl]2-(trimethylazaniumyl)ethyl phosphate (PEIPC) and/or 1-palmitoyl-2-azelaoyl-sn-glycero-3-phosphocholine (PAzePC).

55. A pharmaceutical formulation comprising the composition of any one of claims 1-54, and a pharmaceutically acceptable excipient.

56. A method for production of hyperactivated dendritic cells, the method comprising contacting the dendritic cells with an effective amount of the composition of any one of claims 1-54, any one of claims 25-54, or the formulation of claim 55 to produce hyperactivated dendritic cells, wherein the hyperactivated dendritic cells secrete IL-1beta without undergoing cell death within about 48 hours of exposure.

57. The method of claim 56, wherein the dendritic cells are:
(i) contacted in vivo with the composition; or
(ii) contacted ex vivo with the composition.

58. The method of claim 56 or claim 57, wherein the hyperactivated dendritic cells:
(i) secrete higher levels of one or more of IL-6, TNFα, RANTES and IP-10 than unstimulated dendritic cells or dendritic cells contacted with empty LNPs; and/or
(ii) express higher levels of at least one cell surface marker selected from the group consisting of CCR7, CD40, CD80, CD83, CD86, MHC class IL, MHC class I, and combinations thereof.

59. A pharmaceutical formulation comprising at least 10\hat{ }3, 10\hat{ }4, 10\hat{ }5 or 10\hat{ }6 of the hyperactivated dendritic cells produced by the method of any one of claims 56-58, and a pharmaceutically acceptable excipient.

60. A method of stimulating an immune response against an antigen, comprising administering an effective amount of the pharmaceutical formulation of claim 55 or claim 59 to an individual in need thereof to stimulate the immune response against the antigen.

61. A method of treating cancer, comprising administering an effective amount of the pharmaceutical formulation of claim 55 or claim 59 to an individual in need thereof to treat the cancer.

62. A method of inhibiting abnormal cell proliferation, comprising administering an effective amount of the pharmaceutical formulation of claim 55 or claim 59 to an individual in need thereof to inhibit abnormal cell proliferation.

63. A method of treating or preventing an infectious disease, comprising administering an effective amount of the pharmaceutical formulation of claim 55 to an individual in need thereof to treat or prevent the infectious disease.

64. The method of claim 63, wherein the infectious disease is a viral disease.

65. The method of claim 63, wherein the infectious disease is a bacterial disease.

66. The method or pharmaceutical formulation of any one of claims 56-62, wherein the dendritic cells are mammalian cells.

67. The method or pharmaceutical formulation of claim 66, wherein the mammalian cells are human cells.

68. The method of any one of claims 60-66, wherein the individual is mammal.

69. The method of claim 68, wherein the mammal is a human.

70. The method of claim 68, wherein the mammal is a dog or a cat.

71. The composition, formulation, or method of any one of claims 1-70, wherein the second phospholipid comprises 1,2-distearoyl-sn-glycero-3-phosphocholine (DSPC), and the least one lipid comprises 8-[(2-hydroxyethyl)[6-oxo-6-(undecyloxy)hexyl]amino]-octanoic acid, 1-octylnonyl ester (SM-102) or analogs or derivatives thereof, and cholesterol.

72. The composition, formulation, or method of claim 71, wherein the at least one lipid further comprises a pegylated lipid, optionally wherein the pegylated lipid comprises polyethylene glycol [PEG]2000 dimyristoyl glycerol [DMG].

73. The composition, formulation, or method of any one of claims 1-72, wherein the LNP has an effective diameter of less than about 250 nanometers, optionally from about 25 to about 250 nanometers, optionally from about 50 to about 200 nanometers, or optionally from about 75 to about 175 nanometers.

74. The composition, formulation, or method of claim 73, wherein the LNP has an effective diameter of less than about 200 nanometers.

75. The composition, formulation, or method of claim 73, wherein the LNP has an effective diameter of less than about 150 nanometers.


SEQUENCE LISTING (17 sequences)

SEQ ID NO: 1 - protein, 363 aa, Homo sapiens
PGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 2 - protein, 367 aa, Macaca mulatta
PGARKLRAVLEKLRLSHQDISKAAKVVNGVVGHLLRRLKCEPEFGGVEQLHTGSYYEHVKISAPNEFDVMFKLQVPRIQLEEYSDTGAYYFVKFKRNPEGNPLSLFLEDEILSASKMLSKFRKIIKEEINNIKDVQPDADVIMKRKRPGSPAVTLLINKEISVDITLALELKTSWPASTQEGLAIKNWLSAKVRQQLRLKPFYLVPKHAKEGNGFQEETWRLSFSHIEKEILSNHGKSKTCCENKEMKCCRKDCLKLMKYLLEQLKEKFKDKKHLDKFSSYHVKTAFFHLCTENPQDSQWDPKDLGLCFDNCVTYFLECLRTEQLKNYFIPGFNLFSSNLIDKTSKEFLSKQIEYERNNEFPVFGEF

SEQ ID NO: 3 - protein, 363 aa, Papio anubis
PGARKLRAVLEKLRLSHQDISKAAKVVNGVVGHLLHRLKCESEFEGVEQLHTGSYYEHVKISAPNEFDVMFKLQVPRIQLEEYSDTGAYYFVKFKRNPKGNPLSQFLEDEILSASKMLSQFRKIIKEEINNIKDTDVIMKRKRRGSPAVTLLINKEISVDITLALELKTSWPASTQEGLAIKNWLSAKVRQQLRLKPFYLVPKHAKEGNGFQEETWRLSFSHIEKEILSNHGKSKTCCENKEMKCCRKDCLKLMKYLLEQLKEKFKDKKHLDKFSSYHVKTAFFHLCTENPQDSQWDPKDLGLCFDNCVTHFLQCLRTEKLANYFIPGFNLFSSNLIDKSSKEFLSKQIEYERNNEFPVFGEF

SEQ ID NO: 4 - protein, 363 aa, Nomascus leucogenys
PGARKLRAVLEKLKLSRQEISEAAEVVNGVVDHLLRRLQKCDSEFRGVGLLRTGSYYEHVKISAPNEFDVMFKLEVPRIQLEEYSNTGAYYFVKFKRNPKENPLSQFLEGEILSASKMLSKFRKIIKEEINNIKDTDVIMKRKRGGSPAVTLLISEKISVDITLALESNSNSWPASTQEGLPIKDWLSGKVRRQLRLKPFYLVPKHAKEGNGFQEETWRLSFSHIEKEILNNHGKTKTCCENKNVKCCRKDCLKLMKYLLEQLKERFKDKKHLDKFSSYHVKTAFFHVCTENPQDSQWDPKNLGFCFDKCVIYFLKCLRTEQLGNYFIPGFNLFSSNLIDKRSKEFLSKQIEYERNNEYPVFG

SEQ ID NO: 5 - protein, 365 aa, Hylobates lar
PGARKLPAVLEKLKLSRQEICEAAEVVNGVVDHLLRRLQKCDSEFRGVGLLRTGSYYEHVKISAPNEFDVMFKLEVPRIQLEEYSNTGAYYFVKFKRNPKENPLCQFLEGEILSASKMLSKFRKIIKEEINNIKDTDVILKRKRGGSPAVTLLISEKISVDITLALESKSSSWPASTQEGLPIKDWLSGKVRTQLRLKPFYLVPKHAKEGNGFQEETWRLSFSHIEKEILNNHGETKTCCENKKVKCCRKDCLKLMKYLLEQLKERFKDKKHLDKFSSYHVKTAFFHVCTENPKDSQWDPKDLGLCFDKCVIYFLQCLSTEQLGNYFIPGFNLFSSNLIDKRSKEFLSKQIEYERNNEYPVFGKC

SEQ ID NO: 6 - protein, 364 aa, Pongo abelii
PGASKLRAVLEKLKLSRLEISKAAEVVNRVVDHLLRRLQKYNSEFRGVGLLRTGSYYEHVKISAPNEFDVMFKLEVPRIQLEEYSNTGAYYFVKFKRNPKENPLSQFLEGEILSASKMLSKFRKIIKEEINNIKDTDVIMKRKRGGSPAVTLLISEEISVDITLALESKSSWPASTQEGLPIKNWLSAKVRKQLRLKPFYLVPKHAKEGNGFQEETWRLSFSHIEKEILNNHGKSKTCCEDKEVKCCRKDCLKLMKYLLEQLKERFKDKKHLDKFSSYHVKTAFFHVCTQNPQDSQWDPKDLGLCFDNCVTYFLQCLRTEQLGNYFIPGFNLFSSNLIDKRSKEFLTKQIEYERNNEFPVFDEF

SEQ ID NO: 7 - protein, 361 aa, Pan troglodytes
PGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINNIKDVIMKRKRGGSPAVTLLISEKISVDITLALESKSSWPASTQEGLPIKNWLSAKVRKQLRLKPFYLVPKHAKEGNGFQEETWRLSFSHIEKEILNNHGKSKTCCEIKEAKCCRKDCLKLMKYLLEQLKEKFKDKKHLDKFSSYHVKTAFFHVCTENPQDSQWDRKDLGLCFDNCVTYFLQCLRIEKLENYFIPEFNLFSSDLIDKRSKEFLTKQIEYERNNEFPVFDEF

SEQ ID NO: 8 - protein, 363 aa, Gorilla gorilla
PGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINNIKDTDVIMKRKRGGSPAVTLLISEKISVDITLALESKSSWPASTQEGLRIQNWLSAKVRRQLRLKPFYLVPKHAKEGNGFQEETWRLSFSHIEKEILNNHGKSKTCCENKEAKCCRKDCLKLMKYLLEQLKERFKDKKHLDKFSSYHVKTAFFHVCTQNPQDSQWDRKQLGLCFDNCVTYFLQCLRTERLENYFIPEFNLFSRNLIDKRSKEFLTKQIEYERNNEFPVFDEF

SEQ ID NO: 9 - protein, 369 aa, synthetic construct
PGAXKLXAVLEKLXLSXXXIXXAAXXVXXVVXHLLXRLXXXXXXFXGVXXLXTGSYYEHVKISAPNEFDVMFKLXVPRIQLEEYSXTXAYYFVKFKRNPXXNPLXXFLEXEILSASKMLSXFRKIIKEEINXIKXXXXXXDVIXKRKRXGSPAVTLLIXXXISVDITLALEXXXXSWPASTQEGLXIXXWLSXKVRXQLRLKPFYLVPKHAKEGNGFQEETWRLSFSHIEKEILXNHGXXKTCCEXKXXKCCRKDCLKLMKYLLEQLKEXFKDKKHLDKFSSYHVKTAFFHXCTXNPXDSQWDXKXLGXCFDXCVTXFLXCLXXEXLXNYFIPXFNLFSXXLIDKXSKEFLXKQIEYERNNEXPVFXXX

SEQ ID NO: 10 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 11 - protein, 159 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAA

SEQ ID NO: 12 - protein, 21 aa, Thosea asigna virus
  VARIANT 1..3: note = can be present or absent
GSGEGRGSLLTCGDVEENPGP

SEQ ID NO: 13 - protein, 22 aa, Porcine teschovirus-1
  VARIANT 1..3: note = can be present or absent
GSGATNFSLLKQAGDVEENPGP

SEQ ID NO: 14 - protein, 23 aa, Equine rhinitis A virus
  VARIANT 1..3: note = can be present or absent
GSGQCTNYALLKLAGDVESNPGP

SEQ ID NO: 15 - protein, 25 aa, Foot-and-mouth disease virus
  VARIANT 1..3: note = can be present or absent
GSGVKQTLNFDLLKLAGDVESNPGP

SEQ ID NO: 16 - protein, 8 aa, Gallus gallus
SIINFEKL

SEQ ID NO: 17 - DNA, 1098 bp, synthetic construct
  source 1..1098: mol_type = other DNA
atgcccggcgccagcaagctgagggccgtgctggagaagctgaagctgagcagggacgacatcagcaccgccgccggcatggtgaagggcgtggtggaccacctgctgctgaggctgaagtgcgacagcgccttcaggggcgtgggcctgctgaacaccggcagctactacgagcacgtgaagatcagcgcccccaacgagttcgacgtgatgttcaagctggaggtgcccaggatccagctggaggagtacagcaacaccagggcctactacttcgtgaagttcaagaggaaccccaaggagaaccccctgagccagttcctggagggcgagatcctgagcgccagcaagatgctgagcaagttcaggaagatcatcaaggaggagatcaacgacatcaaggacaccgacgtgatcatgaagaggaagaggggcggcagccccgccgtgaccctgctgatcagcgagaagatcagcgtggacatcaccctggccctggagagcaagagcagctggcccgccagcacccaggagggcctgaggatccagaactggctgagcgccaaggtgaggaagcagctgaggctgaagcccttctacctggtgcccaagcacgccaaggagggcaacggcttccaggaggagacctggaggctgagcttcagccacatcgagaaggagatcctgaacaaccacggcaagagcaagacctgctgcgagaacaaggaggagaagtgctgcaggaaggactgcctgaagctgatgaagtacctgctggagcagctgaaggagaggttcaaggacaagaagcacctggacaagttcagcagctaccacgtgaagaccgccttcttccacgtgtgcacccagaacccccaggacagccagtgggacaggaaggacctgggcctgtgcttcgacaactgcgtgacctacttcctgcagtgcctgaggaccgagaagctggagaactacttcatccccgagttcaacctgttcagcagcaacctgatcgacaagaggagcaaggagttcctgaccaagcagatcgagtacgagaggaacaacgagttccccgtgttcgacgagttctaatga